(19) United States
(12) Patent Application Publication
(10) Pub. No.: US 2018/0149635 A1
(43) Pub. Date: May 31, 2018

(54) SENSOR

(71) Applicants: Digisense Ltd., Petach Tikva (IL); Expro3, LLC., Coral Gables, FL (US)

(72) Inventor: Eyall Abir, Petach Tikva (IL)

(21) Appl. No.: 15/568,987

(22) PCT Filed: Apr. 29, 2016
(86) PCT No.: PCT/US2016/030181

§ 371 Date: Oct. 24, 2017

Related U.S. Application Data
(60) Provisional application No. 62/155,089, filed on Apr. 30, 2015.
(60) Provisional application No. 62/159,814, filed on May 11, 2015.
(60) Provisional application No. 62/164,390, filed on May 20, 2015.
(60) Provisional application No. 62/199,042, filed on Jul. 30, 2015.
(60) Provisional application No. 62/238,426, filed on Oct. 7, 2015.
(60) Provisional application No. 62/298,623, filed on Feb. 23, 2016.
(60) Provisional application No. 62/298,629, filed on Feb. 23, 2016.

(51) Int. Cl. G01N 33/493; G01F 22/00; G01N 21/55; G01N 21/3577; G01N 21/80; G01N 33/487; G01N 33/52; G01N 33/497; G01N 33/483

(52) U.S. Cl. CPC G01N 33/493; G01F 22/00; G01N 21/55; G01N 21/3577; A61F 13/42; G01N 33/48792; G01N 33/528; G01N 33/497; G01N 33/4833; G01N 21/80

(57) ABSTRACT

The present invention provides a urine analyzer comprising: a. at least one discrete test area configured to perform a colorimetric test specific for an analyte selected from the group consisting of glucose, protein, pH, blood, ketone bodies, bilirubin, and any combination thereof, wherein each discrete test area is configured to perform a different colorimetric test; b. at least one color sensor, configured to read the at least one discrete test area, and output an indication based on the presence of the at least one analyte; and c. an integrated circuit, configured to receive an indication from the at least one color sensor, and configured to compute, based on the indication from the at least one color sensor, the amount of at least one analyte selected from the group consisting of glucose, protein, pH, blood, ketone bodies, bilirubin, specific gravity, urobilinogen, nitrite, leuokocytes, blood, and any combination thereof, in the urine.

### CROSS REFERENCE TO RELATED APPLICATIONS

This application claims priority to U.S. Provisional Patent Application Ser. No. 62/155,089, filed on Apr. 30, 2015; U.S. Provisional Patent Application Ser. No. 62/159,814, filed on May 11, 2015; U.S. Provisional Patent Application Ser. No. 62/164,390, filed on May 20, 2015; U.S. Provisional Patent Application Ser. No. 62/199,042, filed on Jul. 30, 2015; U.S. Provisional Patent Application Ser. No. 62/238,426, filed on Oct. 7, 2015; U.S. Provisional Patent Application Ser. No. 62/298,623, filed on Feb. 23, 2016; and U.S. Provisional Patent Application Ser. No. 62/298,629, filed on Feb. 26, 2016, the entire contents of which are hereby incorporated by reference in their entirety.

### FIELD OF THE INVENTION

The present invention generally relates to devices to analyze urine, feces and flatus.

### BACKGROUND

The amount of urine, feces, and/or flatus produced by a subject, the chemical composition of the urine, feces, and/or flatus, and the frequency of urination and/or defecation are indicators of the health of the subject.

### SUMMARY

In one embodiment, the present invention is a urine analyzer comprising:
a urine collection bag; and
a sensor, comprising:
   a. at least one light-emitting diode (LED) configured to illuminate a portion of the urine collection bag;
   b. at least one photodetector configured to output an indication of an amount of light reflected from the portion of the urine collection bag illuminated by the at least one LED; and
   c. an integrated circuit, configured to receive an indication from the at least one photodetector, and configured to compute, based on the indication from the at least one photodetector, a volume of urine in the urine collection bag.

In one embodiment, the present invention is a urine analyzer comprising:
a urine collection bag; and
a sensor, comprising:
   a. at least one light-emitting diode (LED) configured to illuminate a portion of the urine collection bag;
   b. at least one photodetector configured to output an indication of an amount of light reflected from the portion of the urine collection bag illuminated by the at least one LED;
   c. a first analyte sensor configured to detect at least one analyte selected from the group consisting of glucose, protein, pH, blood, ketone bodies, bilirubin, and any combination thereof, and output an indication based on the presence of the at least one analyte; and
   d. an integrated circuit, configured to receive an indication from the at least one photodetector and the first analyte sensor, and
      i. configured to compute, based on the indication from the at least one photodetector, a volume of urine in the urine collection bag; and
      ii. configured to compute, based on the indication from the first analyte sensor, the amount of at least one analyte selected from the group consisting of glucose, protein, pH, blood, ketone bodies, bilirubin, and any combination thereof, in the urine.

In one embodiment, the urine analyzer further comprises:
a. at least one chemical test strip configured to detect at least one analyte selected from the group consisting of glucose, protein, pH, blood, ketone bodies, bilirubin, and any combination thereof; and
b. a second analyte sensor configured to read the at least one chemical test strip, and output an indication based on the presence of the at least one analyte, and wherein the integrated circuit is configured to receive an indication from the at least one photodetector, the first analyte sensor, and the second analyte sensor, and
   i. configured to compute, based on the indication from the at least one photodetector, the hydration level of the subject; and
   ii. configured to compute, based on the indication from the first analyte sensor and/or the second analyte sensor, the amount of at least one analyte selected from the group consisting of glucose, protein, pH, blood, ketone bodies, bilirubin, and any combination thereof, in the urine.

In one embodiment, the present invention is a urine analyzer comprising:
a urine collection bag; and
a sensor comprising:
   a. a fluid reservoir;
   b. at least one light-emitting diode (LED) configured to illuminate a portion of the fluid reservoir;
   c. at least one photodetector configured to output an indication of an amount of light reflected from the portion of the fluid reservoir illuminated by the at least one LED;
   d. at least one chemical test strip configured to detect at least one analyte selected from the group consisting of glucose, protein, pH, blood, ketone bodies, bilirubin, and any combination thereof;
   e. a second analyte sensor configured to read the at least one test strip, and output an indication based on the presence of the at least one analyte; and
   f. an integrated circuit, configured to receive an indication from the at least one photodetector and the second analyte sensor, and
      i. configured to compute, based on the indication from the at least one photodetector, the hydration level of the subject, and
      ii. configured to compute, based on the indication from the second analyte sensor, the amount of at least one analyte selected from the group consisting of glucose, protein, pH, blood, ketone bodies, bilirubin, and any combination thereof, in the urine.

In one embodiment, the present invention is a urine analyzer, comprising:
a. a fluid reservoir;
b. at least one light-emitting diode (LED) configured to illuminate a portion of the fluid reservoir;
c. at least one photodetector configured to output an indication of an amount of light reflected from the portion of the fluid reservoir illuminated by the at least one LED;
d. a first analyte sensor configured to detect at least one analyte selected from the group consisting of glucose, protein, pH, blood, ketone bodies, bilirubin, and any combination thereof, and output an indication based on the presence of the at least one analyte; and
e. an integrated circuit, configured to receive an indication from the at least one photodetector and the first analyte sensor, and
   i. configured to compute, based on the indication from the at least one photodetector, the hydration level of the subject, and
   ii. configured to compute, based on the indication from the first analyte sensor, the amount of at least one analyte selected from the group consisting of glucose, protein, pH, blood, ketone bodies, bilirubin, and any combination thereof, in the urine.

In one embodiment, the urine analyzer further comprises:
a. at least one chemical test strip configured to detect at least one analyte selected from the group consisting of glucose, protein, pH, blood, ketone bodies, bilirubin, and any combination thereof; and
b. a second analyte sensor configured to read the at least one chemical test strip, and output an indication based on the presence of the at least one analyte, and wherein the integrated circuit is configured to receive an indication from the at least one photodetector, the first analyte sensor, and the second analyte sensor, and
   i. configured to compute, based on the indication from the at least one photodetector, the hydration level of the subject; and
   ii. configured to compute, based on the indication from the first analyte sensor and/or the second analyte sensor, the amount of at least one analyte selected from the group consisting of glucose, protein, pH, blood, ketone bodies, bilirubin, and any combination thereof, in the urine.

In one embodiment, the present invention is a urine analyzer, comprising:
a. a fluid reservoir;
b. at least one light-emitting diode (LED) configured to illuminate a portion of the fluid reservoir;
c. at least one photodetector configured to output an indication of an amount of light reflected from the portion of the fluid reservoir illuminated by the at least one LED;
d. at least one chemical test strip configured to detect at least one analyte selected from the group consisting of glucose, protein, pH, blood, ketone bodies, bilirubin, and any combination thereof;
e. a second analyte sensor configured to read the at least one test strip, and output an indication based on the presence of the at least one analyte; and
f. an integrated circuit, configured to receive an indication from the at least one photodetector and the second analyte sensor, and
   i. configured to compute, based on the indication from the at least one photodetector, the hydration level of the subject, and
   ii. configured to compute, based on the indication from the second analyte sensor, the amount of at least one analyte selected from the group consisting of glucose, protein, pH, blood, ketone bodies, bilirubin, and any combination thereof, in the urine.

In one embodiment, the urine analyzer further at least one sensor comprising a thermopile, configured to output an indication of the presence of urine and/or feces by detecting the thermal radiation emitted by the urine and/or feces, and wherein the integrated circuit is configured to receive an indication from the at least one photodetector, the first analyte sensor, the second analyte sensor, and the thermopile and
i. configured to compute, based on the indication from the at least one photodetector, the hydration level of the subject;
ii. configured to compute, based on the indication from the second analyte sensor, the amount of at least one analyte selected from the group consisting of glucose, protein, pH, blood, ketone bodies, bilirubin, and any combination thereof, in the urine; and
iii. configured to compute, based on the indication from the at least one sensor comprising a thermopile, the presence of urine and/or feces.

In one embodiment, the at least one analyte detected by the first analyte sensor is different than the at least one analyte detected by the at least one chemical strip.

In one embodiment, the integrated circuit is configured to compute, based on the indication, the hydration level of the subject.

In one embodiment, the at least one LED is a white-light LED.

In one embodiment, the at least one LED is an infrared LED.

In one embodiment, the at least one photodetector is selected from the group consisting of: photoresistors, photovoltaic cells, photodiodes, infrared sensors, phototransistors, CCDs (charge coupled devices), and CMOS (complementary metal oxide semiconductors).

In one embodiment, the first analyte sensor is a humidity sensor.

In one embodiment, the urine analyzer further comprises a screen operatively coupled to the first integrated circuit, wherein the screen is configured to display the volume of urine in the urine collection bag.

In one embodiment, the urine analyzer further comprises a screen operatively coupled to the first integrated circuit, wherein the screen is configured to display the hydration level of the subject.

In one embodiment, the urine analyzer further comprises a screen operatively coupled to the first integrated circuit, wherein the screen is configured to display the volume of urine collected and the hydration level of the subject.

In one embodiment, the integrated circuit is configured to transmit the computation to a remote device.

In one embodiment, the integrated circuit is configured to transmit the computation to a remote device via a radio transmission.

In one embodiment, the integrated circuit is configured to transmit the computation to a remote device via an audible signal.

In one embodiment, the integrated circuit is configured to transmit the computation to a remote device via a tactile signal.

In one embodiment, the integrated circuit is configured to transmit the computation to a remote device via a visual signal.

In one embodiment, the present invention is a device for detecting flatus, comprising:
a sensor configured to detect flatus and output an indication that flatus has been detected; and
an integrated circuit configured to:
   a. receive the indication from the sensor;
   b. compute, based on the indication, whether flatus is present in the incontinence product, when the flatus was deposited in the incontinence product; and
   c. transmit the computation to a data display unit.

In one embodiment, the present invention is a device for detecting fecal matter, comprising:
a sensor configured to detect fecal matter and output an indication that fecal matter has been detected; and
an integrated circuit configured to:
   a. receive the indication from the sensor;
   b. compute, based on the indication, whether fecal matter is present in the incontinence product, when the fecal matter was deposited in the incontinence product; and
   c. transmit the computation to a data display unit.

In one embodiment, the present invention is a device for detecting flatus and fecal matter, comprising:
a sensor configured to detect flatus and fecal matter and output an indication that flatus and fecal matter has been detected; and
an integrated circuit configured to:
   a. receive the indication from the sensor;
   b. compute, based on the indication, whether flatus, or fecal matter, or both is present in the incontinence product, when the flatus, or fecal matter was deposited in the incontinence product; and
   c. transmit the computation to a data display unit.

In one embodiment, the sensor is a humidity sensor. In one embodiment, the humidity sensor detects flatus. In one embodiment, the humidity sensor detects fecal matter. In one embodiment, the humidity sensor detects flatus and fecal matter.

In one embodiment, the sensor is a volatile organic compound sensor. In one embodiment, the volatile organic compound sensor detects flatus. In one embodiment, the volatile organic compound sensor detects fecal matter. In one embodiment, the volatile organic compound sensor detects flatus and fecal matter.

In one embodiment, the present invention is a device for detecting flatus and fecal matter, comprising:
a first sensor configured to detect flatus and output an indication that flatus has been detected;
a second sensor configured to detect fecal matter and output an indication that fecal matter has been detected, by detecting signals from the first and second sensor; and
an integrated circuit configured to:
   a. receive the indications from the first and second sensor;
   b. compute, based on the indications, whether flatus, or fecal matter, or both is present in the incontinence product, when the flatus, or fecal matter was deposited in the incontinence product; and
   c. transmit the computation to a data display unit.

In one embodiment, the housing further comprises a sticker for external attachment to the article worn by the subject.

In one embodiment, the housing further comprises a VELCRO attachment for external attachment to the article worn by the subject.

In one embodiment, the housing further comprises a clip for external attachment to the article worn by the subject.

In one embodiment, the housing further comprises a screen operative couple to the integrated circuit, wherein the screen is configured to display the amount of urine.

In one embodiment, the housing further comprises a screen operatively coupled to the integrated circuit, wherein the screen is configured to display a signal indicating the presence of flatus, fecal matter, or both flatus and fecal matter.

In one embodiment, the subject is an infant.

In one embodiment, the subject is an adult.

In one embodiment, the present invention is a urine analyzer comprising:
a. at least one discrete test area configured to perform a colorimetric test specific for an analyte selected from the group consisting of glucose, protein, pH, blood, ketone bodies, bilirubin, and any combination thereof, wherein each discrete test area is configured to perform a different colorimetric test;
b. at least one color sensor, configured to read the at least one discrete test area, and output an indication based on the presence of the at least one analyte; and
c. an integrated circuit, configured to receive an indication from the at least one color sensor, and configured to compute, based on the indication from the at least one color sensor, the amount of at least one analyte selected from the group consisting of glucose, protein, pH, blood, ketone bodies, bilirubin, specific gravity, urobilinogen, nitrite, leuokocytes, blood, and any combination thereof, in the urine.

In one embodiment, the urine analyzer is further configured to perform an optical analysis of the urine, wherein an optical sensor is configured to measure the absorbance of the urine and output an indication, based on the absorbance, and the integrated circuit is further configured to receive the indication, and configured to compute the concentration of the urine.

In one embodiment, the at least one discrete test area is incorporated into a test strip.

In one embodiment, the test strip may be removed from the urine analyzer and disposed of.

In one embodiment, every discrete test area has a single color sensor positioned over it, and each color sensor is configured to output an indication based on the presence of the at least one analyte.

In one embodiment, the integrated circuit is further configured to compute, based on the indication from the color sensors positioned over every discrete test area, the amount of at least one analyte selected from the group consisting of glucose, protein, pH, blood, ketone bodies, bilirubin, specific gravity, urobilinogen, nitrite, leuokocytes, blood, and any combination thereof, in the urine.

In one embodiment, the urine analyzer is configured to be integrated into an incontinence device.

In one embodiment, the diaper is configured to allow urine to exit the diaper and enter the urine analyzer.

In one embodiment, the diaper comprises a connector that attaches the urine analyzer to the diaper, and the connector has an orifice, forming a channel through the diaper, that allows urine to exit the diaper and enter the urine analyzer, without being absorbed by the diaper.

In one embodiment, the connector is integrated into the diaper during the manufacturing process of the diaper.

In one embodiment, the connector us added to the diaper after the diaper has been manufactured. In one embodiment, the connector is disposable.

In one embodiment, the diaper is worn by a subject.

In one embodiment, the urine analyzer is configured to be attached to an article used by a subject.

In one embodiment, the article configured to be used by the subject is selected from the group consisting of bedding, and a seat belt latch.

In one embodiment, the subject is an infant.

In one embodiment, the subject is an adult.

In one embodiment, the integrated circuit is configured to transmit the computation to a remote device.

In one embodiment, the integrated circuit is configured to transmit the computation to a remote device via a radio transmission.

In one embodiment, the integrated circuit is configured to transmit the computation to a remote device via an audible signal.

In one embodiment, the integrated circuit is configured to transmit the computation to a remote device via a tactile signal.

In one embodiment, the integrated circuit is configured to transmit the computation to a remote device via a visual signal.

In one embodiment, the urine analyzer further comprises a screen operatively coupled to the integrated circuit, wherein the screen is configured to display the computation.

In one embodiment, urine analyzer further comprises a light-emitting diode (LED) configured to illuminate the at least one discrete area of the test strip.

In one embodiment, the at least one LED is a white-light LED.

In one embodiment, the at least one LED is an infrared LED.

In one embodiment, the present invention is a urine analyzer comprising:
a. at least one discrete test area configured to perform a colorimetric test specific for an analyte selected from the group consisting of glucose, protein, pH, blood, ketone bodies, bilirubin, and any combination thereof, wherein each discrete test area is configured to perform a different colorimetric test;
b. at least one color sensor, configured to read the at least one discrete test area, and output an indication based on the presence of the at least one analyte; and
c. an integrated circuit, configured to receive an indication from the at least one color sensor, and compute, based on the indication from the at least one color sensor, the amount of at least one analyte selected from the group consisting of glucose, protein, pH, blood, ketone bodies, bilirubin, specific gravity, urobilinogen, nitrite, leuokocytes, blood, and any combination thereof, in the urine.

In one embodiment, the urine analyzer is further configured to perform an optical analysis of the urine, wherein an optical sensor is configured to measure the absorbance of the urine and output an indication, based on the absorbance, and the integrated circuit is further configured to receive the indication, and compute the concentration of the urine.

In one embodiment, the at least one discrete test area is incorporated into a test strip.

In one embodiment, the test strip may be removed from the urine analyzer and disposed of.

In one embodiment, every discrete test area has a single color sensor positioned over it, and each color sensor is configured to output an indication based on the presence of the at least one analyte.

In one embodiment, the integrated circuit is further configured to compute, based on the indication from the color sensors positioned over every discrete test area, the amount of at least one analyte selected from the group consisting of glucose, protein, pH, blood, ketone bodies, bilirubin, specific gravity, urobilinogen, nitrite, leuokocytes, blood, and any combination thereof, in the urine.

In one embodiment, the urine analyzer is further configured to locate the integrated circuit remotely from the at least one discrete test area, and the at least one color sensor.

In one embodiment, the urine analyzer is further configured to locate the integrated circuit and the at least one color sensor remotely from the at least one discrete test area.

In one embodiment, the at least one discrete test area, and the at least one color sensor are attached to, or integrated into the inside surface of a diaper, and the integrated circuit is attached to, or integrated into the outside surface of the diaper.

In one embodiment, the integrated circuit is connected to the components of the urine analyzer that are located remotely by a connecting cable.

In one embodiment, the urine analyzer is configured to be integrated into an incontinence device.

In one embodiment, the urine analyzer is configured to be attached to an incontinence device.

In one embodiment, the urine analyzer is configured to be attached to an article worn by the subject.

In one embodiment, the incontinence device is an absorbent pad.

In one embodiment, the absorbent pad is placed under the subject.

In one embodiment, the absorbent pad is placed on the subject's bedding.

In one embodiment, the subject is an infant.

In one embodiment, the subject is an adult.

In one embodiment, the integrated circuit is configured to transmit the computation to a remote device.

In one embodiment, the integrated circuit is configured to transmit the computation to a remote device via a radio transmission.

In one embodiment, the integrated circuit is configured to transmit the computation to a remote device via an audible signal.

In one embodiment, the integrated circuit is configured to transmit the computation to a remote device via a tactile signal.

In one embodiment, the integrated circuit is configured to transmit the computation to a remote device via a visual signal.

In one embodiment, the urine analyzer further comprises a screen operatively coupled to the integrated circuit, wherein the screen is configured to display the computation.

In one embodiment, urine analyzer further comprises a light-emitting diode (LED) configured to illuminate the at least one discrete area of the test strip.

In one embodiment, the at least one LED is a white-light LED.

In one embodiment, the at least one LED is an infrared LED.

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention. The figures are listed below.

### BRIEF DESCRIPTION OF THE DRAWINGS

FIG. 1 shows a system diagram according to some embodiments.

FIG. 2 shows a sensor according to some embodiments.

FIG. 3 shows a diagram of a capacitive humidity sensor according to some embodiments.

FIG. 4 shows a block diagram of a sensor according to some embodiments.

FIG. 5 shows a thermopile according to some embodiments.

FIG. 6 shows a sensor according to some embodiments.

FIG. 7 shows a sensor according to some embodiments.

FIG. 9 shows a sensor according to some embodiments.

FIG. 8 shows an incontinence product with a device according to some embodiments.

FIG. 10 shows a chemical test strip according to some embodiments.

FIG. 11 shows a schematic of a device according to some embodiments.

FIG. 12 shows changes in voltage output from a humidity sensor attached to an incontinence product worn by an adult subject, when the adult subject passed flatus.

FIG. 13 shows changes in voltage output from a humidity sensor attached to an incontinence product worn by an adult subject, when the adult subject defecated.

FIG. 14 shows changes in resistance from a volatile organic compound sensor attached to an incontinence product worn by an infant subject, when the infant subject defecated.

FIG. 15 shows changes in prediction values from a volatile organic compound sensor attached to an incontinence product worn by an infant subject, when the infant subject defecated.

FIG. 16 shows a sensor according to some embodiments.

FIGS. 17 A and B shows a sensor according to some embodiments.

FIG. 18 shows a sensor according to some embodiments.

FIG. 19 shows a sensor according to some embodiments.

FIG. 20 shows a sensor according to some embodiments.

FIG. 21 shows a sensor according to some embodiments.

FIG. 22 shows a sensor according to some embodiments.

FIG. 23 shows a block diagram of a sensor according to some embodiments.

FIG. 24 shows a sensor according to some embodiments.

FIG. 25 shows a sensor and connector according to some embodiments.

FIG. 26 shows a block diagram according to some embodiments.

FIG. 27 shows typical recordings obtained from a thermopile according to some embodiments.

FIG. 28 shows changes in resistance from a volatile organic compound sensor attached to an incontinence product worn by an infant subject, over time, in a clean incontinence product.

FIG. 29 shows changes in prediction values from a volatile organic compound sensor attached to an incontinence product worn by an infant subject, over time, in a clean incontinence product.

FIG. 30 shows changes in resistance from a volatile organic compound sensor attached to an incontinence product worn by an infant subject, over time, in a soiled incontinence product.

FIG. 31 shows changes in resistance from a volatile organic compound sensor attached to an incontinence product worn by an infant subject, over time, in a clean incontinence product.

FIG. 32 shows the change on color of a reagent panel in a sensor according to some embodiments of the present invention, when the subject urinated (A), or in a clean diaper (B).

### DETAILED DESCRIPTION

For clarity of disclosure, and not by way of limitation, the detailed description of the invention is divided into the following subsections that describe or illustrate certain features, embodiments or applications of the present invention.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

### Sensor According to Some Embodiments of the Present Invention

In some embodiments, the present invention includes devices and methods useful for collecting and analyzing urine from a subject. According to certain embodiments, the frequency of urination, the volume of urine, the hydration level, the levels of at least one analyte selected from the group consisting of glucose, protein, pH, blood, ketone bodies, bilirubin, or any combination thereof, is an indication of the health of the subject.

In some embodiments, the present invention is a urine analyzer comprising:
a urine collection bag; and
a sensor, comprising:
   d. at least one light-emitting diode (LED) configured to illuminate a portion of the urine collection bag;
   e. at least one photodetector configured to output an indication of an amount of light reflected from the portion of the urine collection bag illuminated by the at least one LED; and
   f. an integrated circuit, configured to receive an indication from the at least one photodetector, and configured to compute, based on the indication from the at least one photodetector, a volume of urine in the urine collection bag.

In some embodiments, the present invention is a urine analyzer comprising:
a urine collection bag; and
a sensor, comprising:
   e. at least one light-emitting diode (LED) configured to illuminate a portion of the urine collection bag;
   f. at least one photodetector configured to output an indication of an amount of light reflected from the portion of the urine collection bag illuminated by the at least one LED;
   g. a first analyte sensor configured to detect at least one analyte selected from the group consisting of glucose, protein, pH, blood, ketone bodies, bilirubin, and any combination thereof, and output an indication based on the presence of the at least one analyte; and
   h. an integrated circuit, configured to receive an indication from the at least one photodetector and the first analyte sensor, and
      i. configured to compute, based on the indication from the at least one photodetector, a volume of urine in the urine collection bag; and
      ii. configured to compute, based on the indication from the first analyte sensor, the amount of at least one analyte selected from the group consisting of glucose, protein, pH, blood, ketone bodies, bilirubin, and any combination thereof, in the urine.

In some embodiments, the urine analyzer further comprises:
c. at least one chemical test strip configured to detect at least one analyte selected from the group consisting of glucose, protein, pH, blood, ketone bodies, bilirubin, and any combination thereof; and
d. a second analyte sensor configured to read the at least one chemical test strip, and output an indication based on the presence of the at least one analyte, and wherein the integrated circuit is configured to receive an indication from the at least one photodetector, the first analyte sensor, and the second analyte sensor, and
   i. configured to compute, based on the indication from the at least one photodetector, the hydration level of the subject; and
   ii. configured to compute, based on the indication from the first analyte sensor and/or the second analyte sensor, the amount of at least one analyte selected from the group consisting of glucose, protein, pH, blood, ketone bodies, bilirubin, and any combination thereof, in the urine.

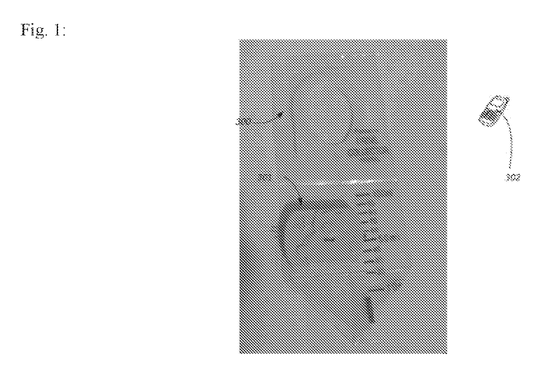

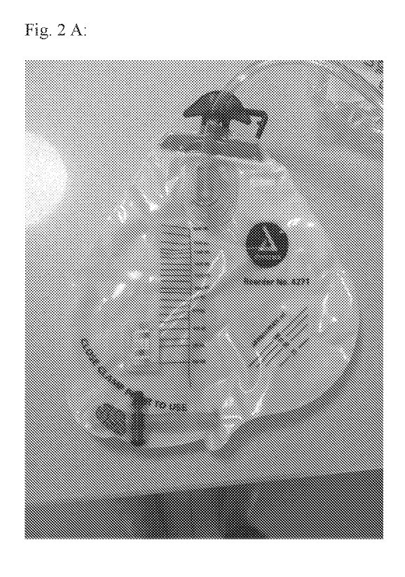

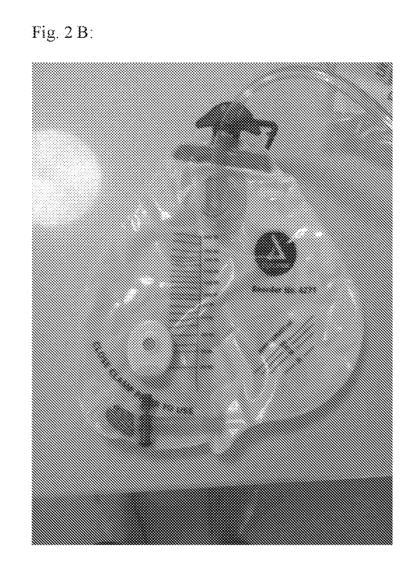

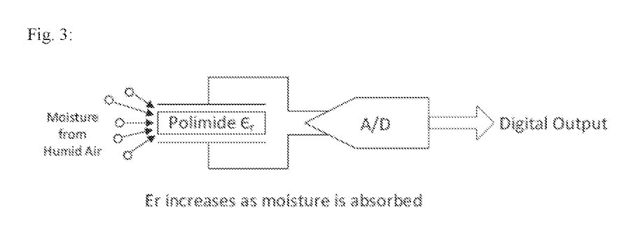

Referring to FIGS. 1 and 2, in some embodiments, the present invention is a urine analyzer comprising:
a urine collection bag; and
a sensor comprising:
   a. a fluid reservoir;
   b. at least one light-emitting diode (LED) configured to illuminate a portion of the fluid reservoir;
   c. at least one photodetector configured to output an indication of an amount of light reflected from the portion of the fluid reservoir illuminated by the at least one LED;
   d. at least one chemical test strip configured to detect at least one analyte selected from the group consisting of glucose, protein, pH, blood, ketone bodies, bilirubin, and any combination thereof;
   e. a second analyte sensor configured to read the at least one test strip, and output an indication based on the presence of the at least one analyte; and
   f. an integrated circuit, configured to receive an indication from the at least one photodetector and the second analyte sensor, and
      i. configured to compute, based on the indication from the at least one photodetector, the hydration level of the subject, and
      ii. configured to compute, based on the indication from the second analyte sensor, the amount of at least one analyte selected from the group consisting of glucose, protein, pH, blood, ketone bodies, bilirubin, and any combination thereof, in the urine.

In some embodiments, the present invention is a urine analyzer, comprising:
f. a fluid reservoir;
g. at least one light-emitting diode (LED) configured to illuminate a portion of the fluid reservoir;
h. at least one photodetector configured to output an indication of an amount of light reflected from the portion of the fluid reservoir illuminated by the at least one LED;
i. a first analyte sensor configured to detect at least one analyte selected from the group consisting of glucose, protein, pH, blood, ketone bodies, bilirubin, and any combination thereof, and output an indication based on the presence of the at least one analyte; and
j. an integrated circuit, configured to receive an indication from the at least one photodetector and the first analyte sensor, and
   i. configured to compute, based on the indication from the at least one photodetector, the hydration level of the subject, and
   ii. configured to compute, based on the indication from the first analyte sensor, the amount of at least one analyte selected from the group consisting of glucose, protein, pH, blood, ketone bodies, bilirubin, and any combination thereof, in the urine.

In some embodiments, the urine analyzer further comprises:
c. at least one chemical test strip configured to detect at least one analyte selected from the group consisting of glucose, protein, pH, blood, ketone bodies, bilirubin, and any combination thereof; and
d. a second analyte sensor configured to read the at least one chemical test strip, and output an indication based on the presence of the at least one analyte, and wherein the integrated circuit is configured to receive an indication from the at least one photodetector, the first analyte sensor, and the second analyte sensor, and
   i. configured to compute, based on the indication from the at least one photodetector, the hydration level of the subject; and
   ii. configured to compute, based on the indication from the first analyte sensor and/or the second analyte sensor, the amount of at least one analyte selected from the group consisting of glucose, protein, pH, blood, ketone bodies, bilirubin, and any combination thereof, in the urine.

In some embodiments, the present invention is a urine analyzer, comprising:
g. a fluid reservoir;
h. at least one light-emitting diode (LED) configured to illuminate a portion of the fluid reservoir;
i. at least one photodetector configured to output an indication of an amount of light reflected from the portion of the fluid reservoir illuminated by the at least one LED;
j. at least one chemical test strip configured to detect at least one analyte selected from the group consisting of glucose, protein, pH, blood, ketone bodies, bilirubin, and any combination thereof;
k. a second analyte sensor configured to read the at least one test strip, and output an indication based on the presence of the at least one analyte; and
l. an integrated circuit, configured to receive an indication from the at least one photodetector and the second analyte sensor, and
   iii. configured to compute, based on the indication from the at least one photodetector, the hydration level of the subject, and
   iv. configured to compute, based on the indication from the second analyte sensor, the amount of at least one analyte selected from the group consisting of glucose, protein, pH, blood, ketone bodies, bilirubin, and any combination thereof, in the urine.

In some embodiments, the urine analyzer further at least one sensor comprising a thermopile, configured to output an indication of the presence of urine and/or feces by detecting the thermal radiation emitted by the urine and/or feces, and wherein the integrated circuit is configured to receive an indication from the at least one photodetector, the first analyte sensor, the second analyte sensor, and the thermopile and
iv. configured to compute, based on the indication from the at least one photodetector, the hydration level of the subject;
v. configured to compute, based on the indication from the second analyte sensor, the amount of at least one analyte selected from the group consisting of glucose, protein, pH, blood, ketone bodies, bilirubin, and any combination thereof, in the urine; and
vi. configured to compute, based on the indication from the at least one sensor comprising a thermopile, the presence of urine and/or feces.

In some embodiments, the at least one analyte detected by the first analyte sensor is different than the at least one analyte detected by the at least one chemical strip.

In some embodiments, the integrated circuit is configured to compute, based on the indication, the hydration level of the subject.

In some embodiments, the at least one LED is a white-light LED.

In some embodiments, the at least one LED is an infrared LED.

In some embodiments, the at least one photodetector is selected from the group consisting of: photoresistors, photovoltaic cells, photodiodes, infrared sensors, phototransistors, CCDs (charge coupled devices), and CMOS (complementary metal oxide semiconductors).

In some embodiments, the first analyte sensor is a humidity sensor. An example of a humidity sensor according to some embodiments of the present invention is shown in FIG. 3.

In some embodiments, the urine analyzer further comprises a screen operatively coupled to the first integrated circuit, wherein the screen is configured to display the volume of urine in the urine collection bag.

In some embodiments, the urine analyzer further comprises a screen operatively coupled to the first integrated circuit, wherein the screen is configured to display the hydration level of the subject.

In some embodiments, the urine analyzer further comprises a screen operatively coupled to the first integrated circuit, wherein the screen is configured to display the volume of urine collected and the hydration level of the subject.

In some embodiments, the integrated circuit is configured to transmit the computation to a remote device.

In some embodiments, the integrated circuit is configured to transmit the computation to a remote device via a radio transmission.

In some embodiments, the integrated circuit is configured to transmit the computation to a remote device via an audible signal.

In some embodiments, the integrated circuit is configured to transmit the computation to a remote device via a tactile signal.

In some embodiments, the integrated circuit is configured to transmit the computation to a remote device via a visual signal.

In some embodiments, the urine analyzer is configured to be integrated into an incontinence device.

In some embodiments, the urine analyzer is configured to be attached to an incontinence device.

In some embodiments, the urine analyzer is configured to be attached to an article worn by the subject.

In some embodiments, the incontinence device is an absorbent pad.

In some embodiments, the absorbent pad is placed under the subject.

In some embodiments, the absorbent pad is placed on the subject's bedding.

In some embodiments, the subject is an infant.

In some embodiments, the subject is an adult.

In some embodiments, the present invention is a urine analyzer comprising: a urine collection bag configured to be attached to a subject, wherein the urine collection bag comprises: a sensor comprising: (i) a light-emitting diode (LED) configured to illuminate a portion of the urine collection bag; (ii) a photodetector configured to output an indication of an amount of light reflected from the portion of the urine collection bag illuminated by the LED; (iii) a first analyte sensor configured detect at least one analyte selected from the group consisting of glucose, protein, pH, blood, ketone bodies, bilirubin, and any combination thereof, and output an indication based on the presence of the at least one analyte; and (iv) an integrated circuit, configured to receive an indication from the photodetector and the first analyte sensor, and compute, (i) based on the indication from the photodetector, a volume of urine in the urine collection bag, and (ii) compute, based on the indication from the first analyte sensor, the amount of at least one analyte selected from the group consisting of glucose, protein, pH, blood, ketone bodies, bilirubin, and any combination thereof, in the urine.

In some embodiments, the urine analyzer further comprises (i) at least one chemical test strip configured to detect at least one analyte selected from the group consisting of glucose, protein, pH, blood, ketone bodies, bilirubin, and any combination thereof, and (ii) a second analyte sensor configured to read the at least one chemical test strip, and output an indication based on the presence of the at least one analyte, and wherein the integrated circuit is configured to receive an indication from the photodetector, the first analyte sensor, and the second analyte sensor, and compute, (i) based on the indication from the photodetector, the hydration level of the subject, and (ii) compute, based on the indication from the first analyte sensor and or the second analyte sensor, the amount of at least one analyte selected from the group consisting of glucose, protein, pH, blood, ketone bodies, bilirubin, and any combination thereof, in the urine.

In some embodiments, the at least one analyte detected by the first analyte sensor is different than the at least one analyte detected by the at least one chemical strip.

In some embodiments, the present invention is a urine analyzer comprising: a urine collection bag configured to be attached to an incontinence device worn by a subject, wherein the urine collection bag comprises: a sensor comprising: (i) a fluid reservoir; (ii) a light-emitting diode (LED) configured to illuminate a portion of the fluid reservoir; (iii) a photodetector configured to output an indication of an amount of light reflected from the portion of the fluid reservoir illuminated by the LED; (iv) at least one chemical test strip configured to detect at least one analyte selected from the group consisting of glucose, protein, pH, blood, ketone bodies, bilirubin, and any combination thereof; (v) a second analyte sensor configured to read the at least one test strip, and output an indication based on the presence of the at least one analyte; and (vi) an integrated circuit, configured to receive an indication from the photodetector and the second analyte sensor, and compute, (i) based on the indication from the photodetector, the hydration level of the subject, and (ii) compute, based on the indication from the second analyte sensor, the amount of at least one analyte selected from the group consisting of glucose, protein, pH, blood, ketone bodies, bilirubin, and any combination thereof, in the urine.

In some embodiments, the present invention is a urine analyzer comprising: a urine collection bag configured to be attached to a subject, wherein the urine collection bag comprises: a sensor comprising: (i) at least one chemical test strip configured to detect at least one analyte selected from the group consisting of glucose, protein, pH, blood, ketone bodies, bilirubin, and any combination thereof; (ii) a second analyte sensor configured to read the at least one test strip, and output an indication based on the presence of the at least one analyte; and (iii) an integrated circuit, configured to receive an indication from the second analyte sensor, and compute, based on the indication from the second analyte sensor, the amount of at least one analyte selected from the group consisting of glucose, protein, pH, blood, ketone bodies, bilirubin, and any combination thereof, in the urine.

In some embodiments, the urine analyzer further comprises at least one sensor comprising a thermopile, configured to output an indication of the presence of urine and/or feces by detecting the thermal radiation emitted by the urine and/or feces, and wherein the integrated circuit is configured to receive an indication from the photodetector, the first analyte sensor, the second analyte sensor, and the thermopile and compute, (i) based on the indication from the photodetector, the hydration level of the subject, (ii) compute, based on the indication from the first analyte sensor and/or the second analyte sensor, the amount of at least one analyte selected from the group consisting of glucose, protein, pH, blood, ketone bodies, bilirubin, and any combination thereof, in the urine, and (iii) compute, based on the indication from the thermopile, the presence of urine and/or feces.

In some embodiments, the present invention is a urine analyzer configured to be attached to an incontinence device worn by a subject, wherein the urine analyzer comprises: a sensor comprising: (i) a fluid reservoir; (ii) a light-emitting diode (LED) configured to illuminate a portion of the fluid reservoir; (iii) a photodetector configured to output an indication of an amount of light reflected from the portion of the fluid reservoir illuminated by the LED; (iv) a first analyte sensor configured to detect at least one analyte selected from the group consisting of glucose, protein, pH, blood, ketone bodies, bilirubin, and any combination thereof, and output an indication based on the presence of the at least one analyte; and (v) an integrated circuit, configured to receive an indication from the photodetector and the first analyte sensor, and compute, (i) based on the indication from the photodetector, the hydration level of the subject, and (ii) compute, based on the indication from the first analyte sensor, the amount of at least one analyte selected from the group consisting of glucose, protein, pH, blood, ketone bodies, bilirubin, and any combination thereof, in the urine.

In some embodiments, the urine analyzer further comprises (i) at least one chemical test strip configured to detect at least one analyte selected from the group consisting of glucose, protein, pH, blood, ketone bodies, bilirubin, and any combination thereof, and (ii) a second analyte sensor configured to read the at least one chemical test strip, and output an indication based on the presence of the at least one analyte, and wherein the integrated circuit is configured to receive an indication from the photodetector, the first analyte sensor, and the second analyte sensor, and compute, (i) based on the indication from the photodetector, the hydration level of the subject, and (ii) compute, based on the indication from the first analyte sensor and or the second analyte sensor, the amount of at least one analyte selected from the group consisting of glucose, protein, pH, blood, ketone bodies, bilirubin, and any combination thereof, in the urine.

In some embodiments, the at least one analyte detected by the first analyte sensor is different than the at least one analyte detected by the at least one chemical strip.

In some embodiments, the present invention is a urine analyzer comprising: a urine analyzer configured to be attached to an incontinence device worn by a subject, wherein the urine analyzer comprises: a sensor comprising: (i) a fluid reservoir; (ii) a light-emitting diode (LED) configured to illuminate a portion of the fluid reservoir; (iii) a photodetector configured to output an indication of an amount of light reflected from the portion of the fluid reservoir illuminated by the LED; (iv) at least one chemical test strip configured to detect at least one analyte selected from the group consisting of glucose, protein, pH, blood, ketone bodies, bilirubin, and any combination thereof; (v) a second analyte sensor configured to read the at least one test strip, and output an indication based on the presence of the at least one analyte; and (vi) an integrated circuit, configured to receive an indication from the photodetector and the second analyte sensor, and compute, (i) based on the indication from the photodetector, the hydration level of the subject, and (ii) compute, based on the indication from the second analyte sensor, the amount of at least one analyte selected from the group consisting of glucose, protein, pH, blood, ketone bodies, bilirubin, and any combination thereof, in the urine.

In some embodiments, the present invention is a urine analyzer configured to be attached to an incontinence device worn by a subject, wherein the urine analyzer comprises: a sensor comprising: (i) at least one chemical test strip configured to detect at least one analyte selected from the group consisting of glucose, protein, pH, blood, ketone bodies, bilirubin, and any combination thereof; (ii) a second analyte sensor configured to read the at least one test strip, and output an indication based on the presence of the at least one analyte; and (iii) an integrated circuit, configured to receive an indication from the second analyte sensor, and compute, based on the indication from the second analyte sensor, the amount of at least one analyte selected from the group consisting of glucose, protein, pH, blood, ketone bodies, bilirubin, and any combination thereof, in the urine.

In some embodiments, the urine analyzer further comprises a screen operatively coupled to the integrated circuit, wherein the screen is configured to display the hydration level of the subject. The photodetector detects the shade of the urine, which is known to be indicative of the level of hydration. Generally, the darker the urine, the more dehydrated the subject is, and vice versa. Transparent urine usually means a good hydration level. Optionally, the sensor also measures the amount of urine secreted, so that it estimates the level of hydration using a combination of two factors: amount and shade.

In some embodiments, the sensor is attached to a urine collection bag. In some embodiments, a photodetector may be used in the sensor to detect and measure the amount of urine in the collection bag. In some embodiments, an analyte sensor may be used to detect at least one analyte selected from the group consisting of glucose, protein, pH, blood, ketone bodies, bilirubin, and any combination thereof. In some embodiments, the sensor further comprises a screen operatively coupled to the integrated circuit, wherein the screen is configured to display the amount of the at least one analyte present in the urine sample.

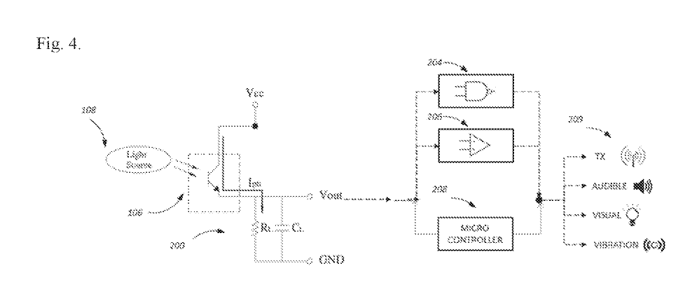

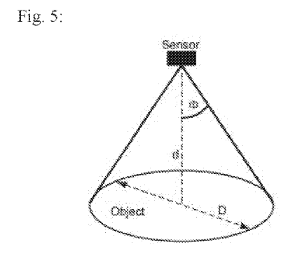

A urine analyzer according to some embodiments is shown in FIG. 4. In the embodiment shown in FIG. 4, a light source 108 passes through a portion of the urine collection bag and illuminates a photodetector 106. In some embodiments, the light source 108 is a white-light LED. In some embodiments, the light source 108 is an infrared LED. The photodetector 106 outputs a voltage, based on the intensity of the light illuminating the photodetector 106, which is received by a microcontroller 208. The microcontroller 208 is configured to process the output signal from the photodetector 106, to transmit a signal 209 specific for the volume of urine, the hydration level of the subject, or both. In the embodiments, the signal can be visual, audible, or tactile. In the embodiments, the signal can be displayed on the sensor, or, alternatively, transmitted to a remote receiver or device.

Non-limiting examples of photodetectors include photoresistors (LDRs), photovoltaic cells, photodiodes (operative in photovoltaic mode or photoconductive mode), IR sensor, phototransistors, CCD (charge coupled device), CMOS (complementary metal oxide semiconductor) and the like.

In some embodiments, the photodetector is the photodetector disclosed in PCT Publication No. WO 2014/064680, the contents of which are incorporated by reference in its entirety.

In some embodiments, the first analyte sensor is a humidity sensor. In certain embodiments, the humidity sensor is a capacitive sensor that consists of two electrodes, separated by a dielectric. In the embodiments, changes in humidity are detected by either a change in the capacitance of the dielectric. Non-limiting examples of humidity sensors suitable for use in the present invention include the P/N:HIH6030 sensor sold by Honeywell.

In some embodiments, the humidity sensor is a resistive sensor that consists of two electrodes, separated by a conductive layer. In the embodiments, changes in humidity are detected by either a change in the conductance of the conductive layer.

In some embodiments, the first analyte sensor is a volatile organic compound sensor. In certain embodiments, the volatile organic compound sensor is a MOS sense element, capable of detecting more than one gaseous compound, including, for example, CO and volatile organic compounds. The presence of a volatile organic compound alters the resistance of the sensor, that generates a signal that can be decoded, using an integrated circuit into a readout of parts per million of a specific volatile organic compound. Non-limiting examples of volatile organic compound sensors suitable for use in the present invention include the AMS P/N: iAQ-core sensor, and the AMS P/N: AS-MLV-P2 sensor.

In some embodiments, the thermopile is a MEMS thermopile. In some embodiments, the MEMS thermopile is a TMP006 thermopile.

In some embodiments, the thermopile is configured to be attached to the external surface of the incontinence device, and monitors an area immediately adjacent to the thermopile. An Example of this is shown in FIG. 5.

The microcontroller 208 is configured to process the output signal from the humidity sensor, to transmit a signal 209 specific for the volume of urine, the hydration level of the subject, or both. In the embodiments, the signal can be visual, audible, or tactile. In the embodiments, the signal can be displayed on the sensor, or, alternatively, transmitted to a remote receiver or device.

In certain embodiments, the urine collection bag is further configured to include an additional sensor, selected from the group consisting of: a Hall Effect detector, an acoustic detector, a magnetic detector, a color detector, a gyro, a tilt sensor, an accelerometer, and a pressure sensor.

In some embodiments, an alternative to the microcontroller 208 comprises a voltage comparator 206 configured to compare the signal received from sensor and to perform comparing to known reference threshold, determining the signal 209.

In some embodiments, an alternative to the microcontroller 208 comprises a discrete electronic circuit 204 configured to test logic level of the output signal received from sensor and determining the signal 209.

Non-limiting examples of a sensor configured to measure the volume of urine and/or the hydration level, and methods to calculate the volume of urine, and/or hydration level of a subject are disclosed in PCT Publication No. WO 2014/064680, the contents of which are incorporated by reference in its entirety.

In some embodiments, the urine analyzer is attached to an incontinence device worn by a subject. In some embodiments, the sensor is integrated into an incontinence device worn by a subject. In some embodiments, a photodetector may be used in the urine analyzer to detect and measure the amount of urine in the incontinence device. In some embodiments, a photodetector may be used in the urine analyzer to determine the hydration level of the subject. In some embodiments, a first analyte sensor may be used to detect at least one analyte selected from the group consisting of glucose, protein, pH, blood, ketone bodies, bilirubin, and any combination thereof. In some embodiments, the device further comprises a screen operatively coupled to the integrated circuit, wherein the screen is configured to display the amount of the at least one analyte present in the urine sample.

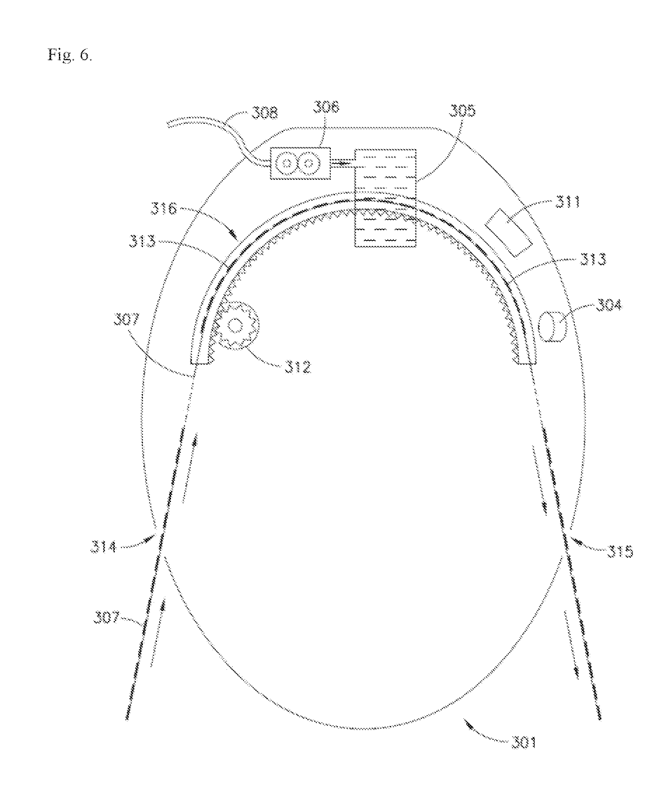

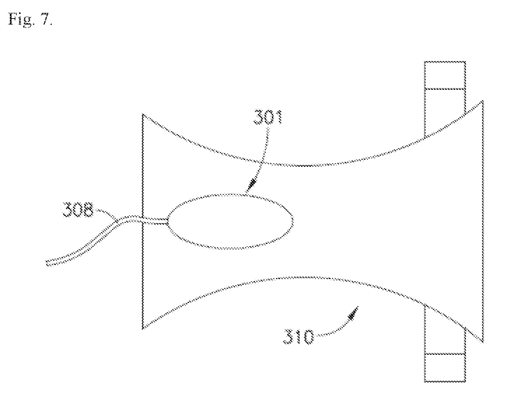

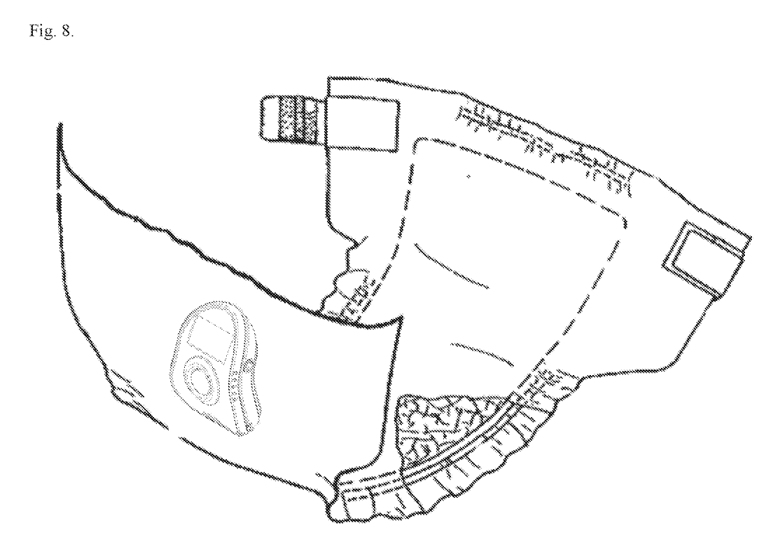

A urine analyzer according to some embodiments is shown in FIGS. 6, 7, and 8. In the embodiment shown in FIG. 6, sensor 301 is configured to be attached to an incontinence device. Tube 308 is fluidly connected to sensor 301 and the incontinence device, wherein the tube 308 collects urine from the incontinence device, using pump 306, and stores a sample of urine in reservoir 305. A chemical test strip 307, comprising discrete test areas 313 is inserted into sensor 301 through a hole 314, and is guided past reservoir 305, heating element 311, and second analyte sensor 304, on a guide rail 316, using a motor 312. Heating element 311 dries chemical test strip 307. Chemical test strip 307 moves past second analyte sensor 304, which reads each discrete test area 313 on chemical test strip 307, prior to chemical test strip 307 being expelled from sensor 301, via hole 315.

In some embodiments, the second analyte sensor configured to read the at least one test strip is a color sensor or an RGB sensor. The choice of RGB sensor is readily determined by one of skill in the art. Non-limiting examples include the RGB sensor manufactured by AMS, and has the part number TCS3471, and the RGB sensor manufactured by Intersil, and has the part number ISL29125.

In some embodiments, discrete test areas 313 contain reagents specific for a given analyte, and undergo a color change when urine contains the specific analyte. Second Analyte sensor 304 detects the color change and outputs a voltage, based on the color change, which is received by a microcontroller. The microcontroller is configured to process the output signal from the second analyte sensor 304, to transmit a signal specific for the specific analyte. In some embodiments, the signal can be visual, audible, or tactile. In some embodiments, the signal can be displayed on the sensor, or, alternatively, transmitted to a remote receiver or device.

One, or more than one chemical test strip may be inserted into sensor 301. The chemical strip may be stored within sensor 301, prior to testing. One, or more than one chemical test strips may be inserted into sensor 301 manually or automatically. Second analyte sensor 304 may read one, or more than one chemical test strip at a time. Second analyte sensor 304 may read one, or more than one discrete test area 313 at a time.

One, or more than one chemical test strips may be stored in an external cartridge and one, or more than one chemical test strip may be inserted into sensor 301.

The chemical test strips expelled from sensor 301 may be collected in an external cartridge.

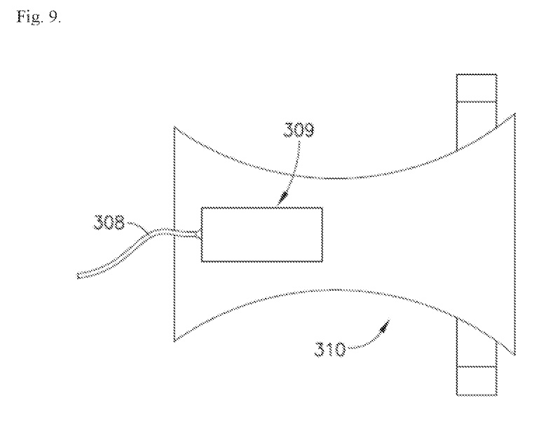

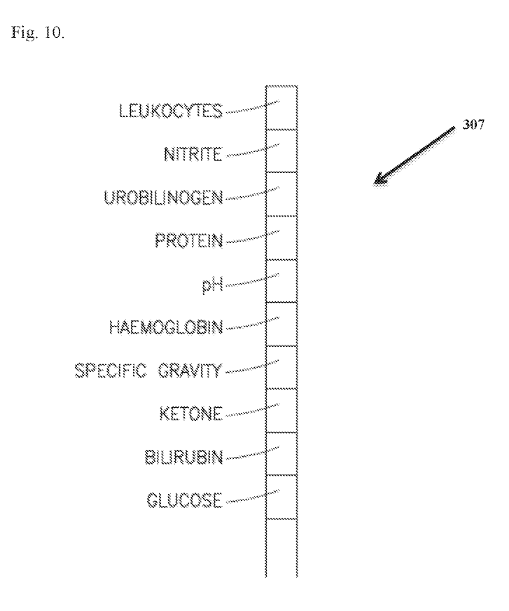

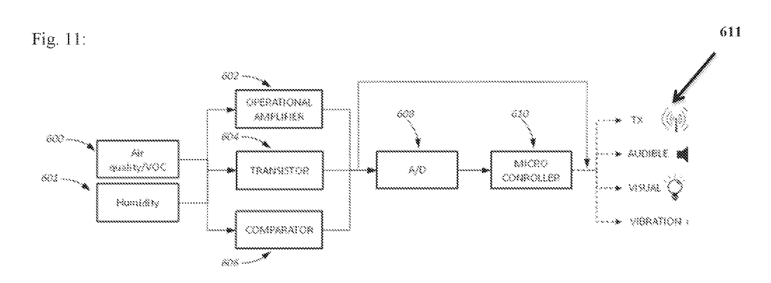

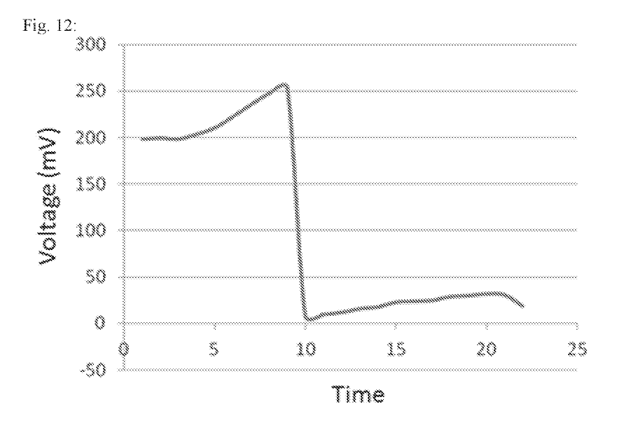

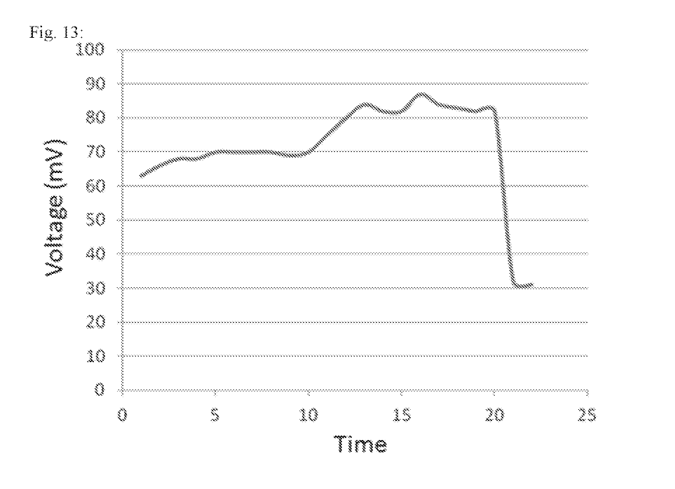

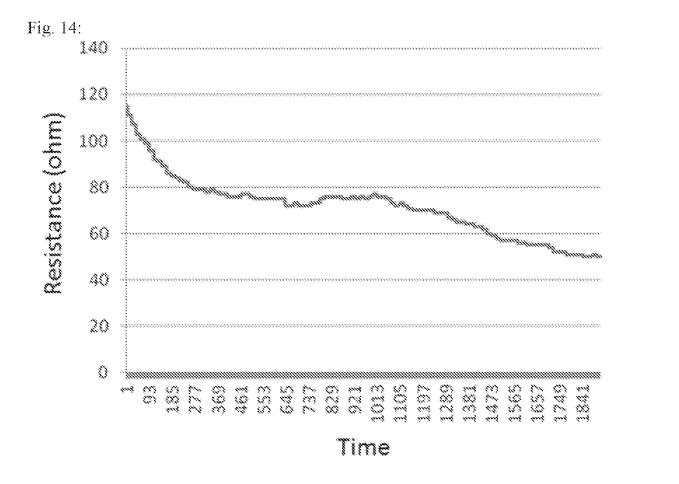

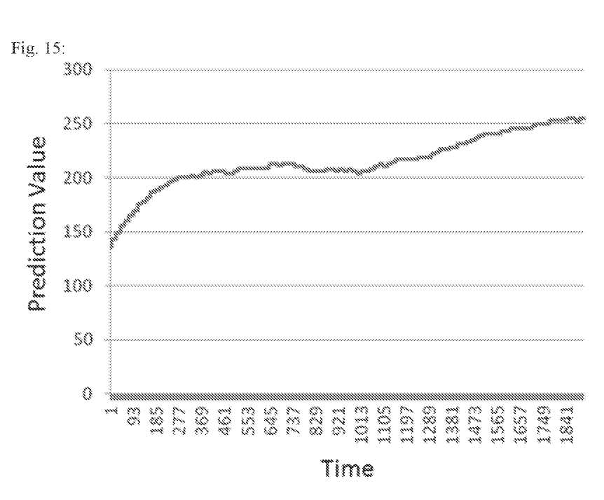

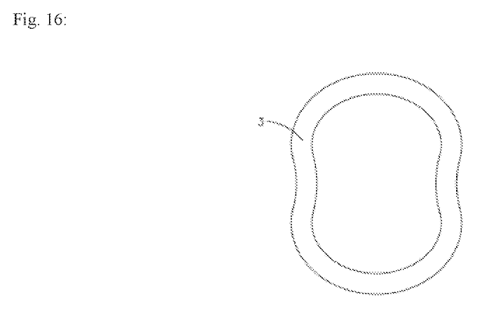

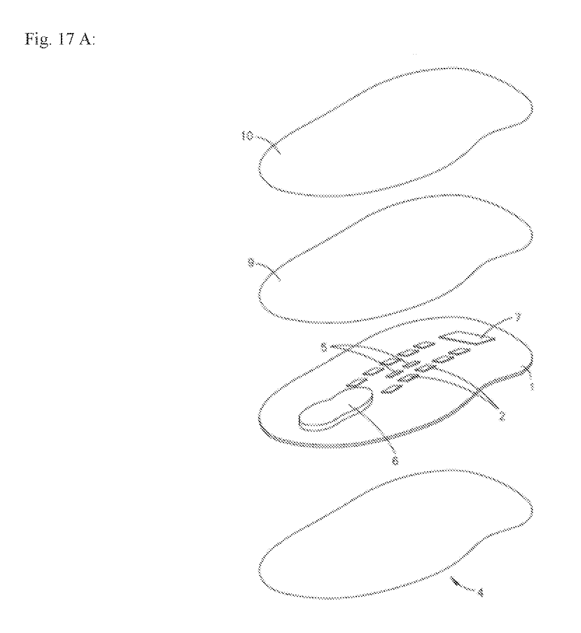

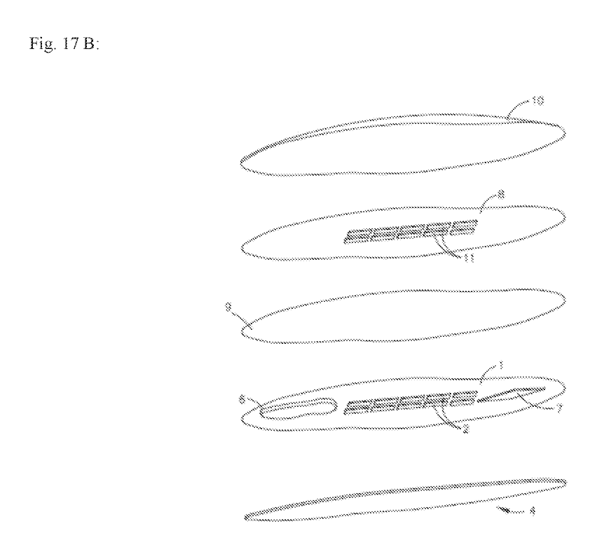

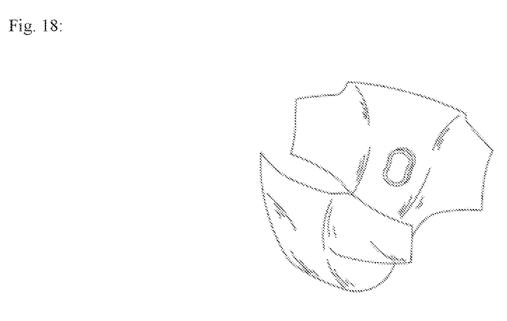

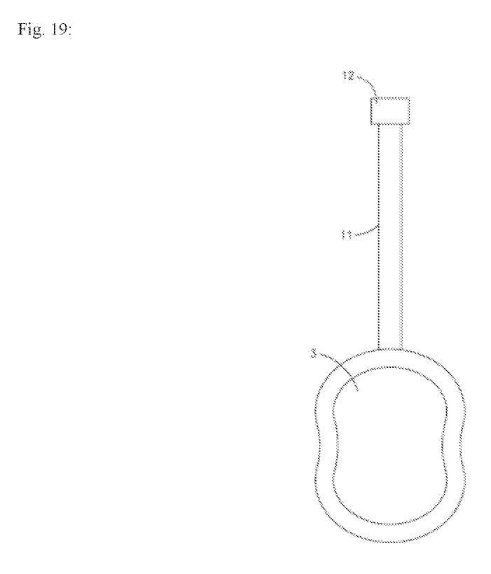

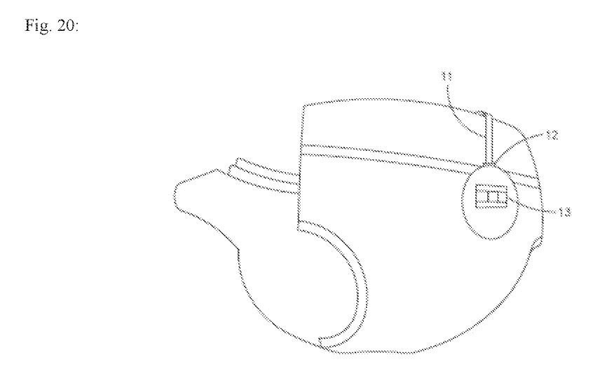

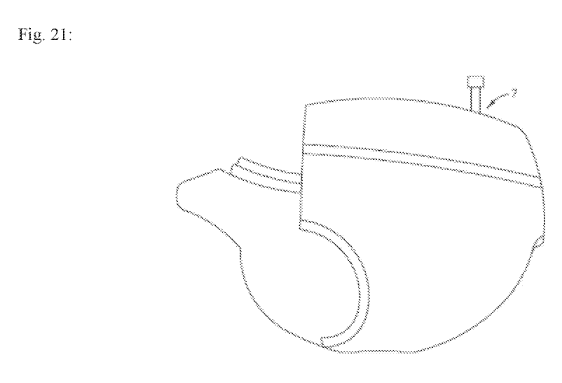

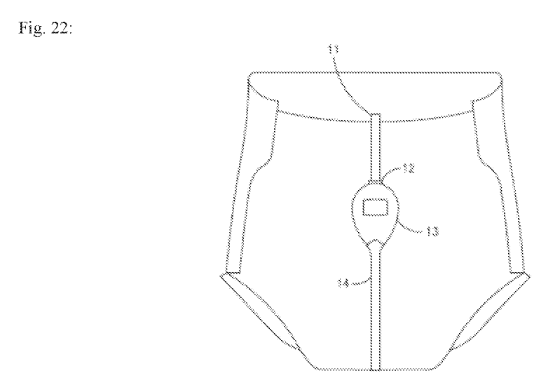

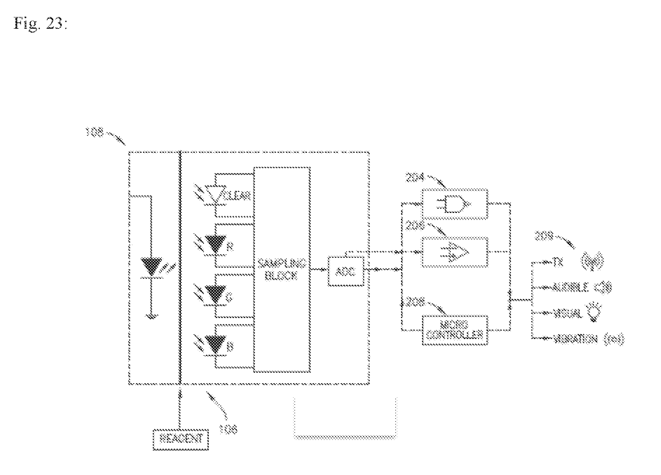

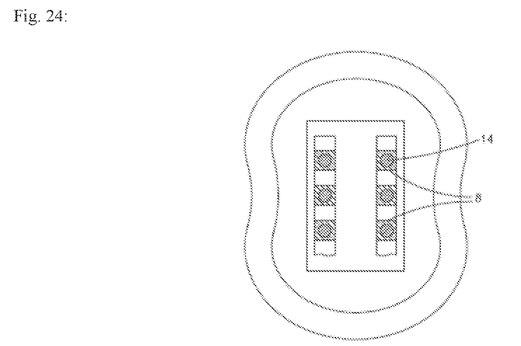

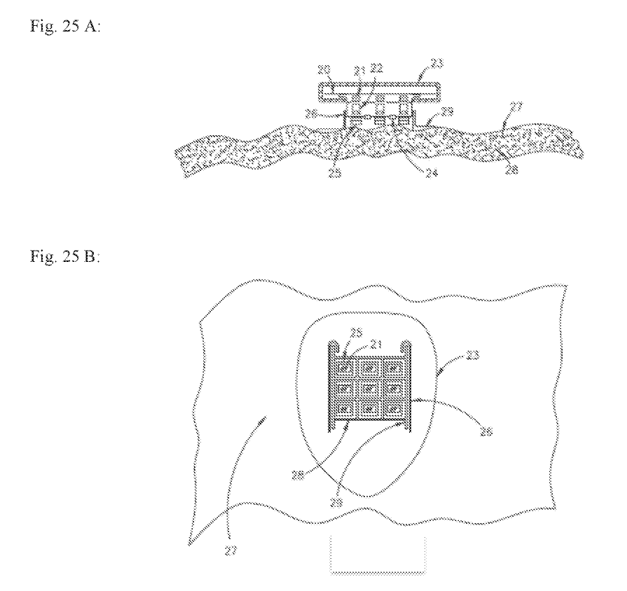

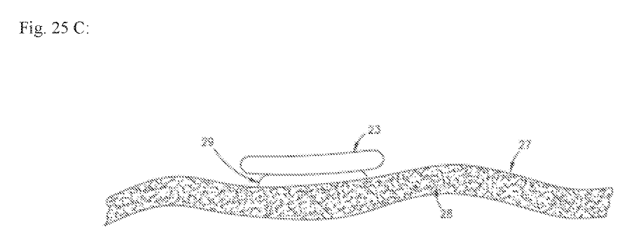

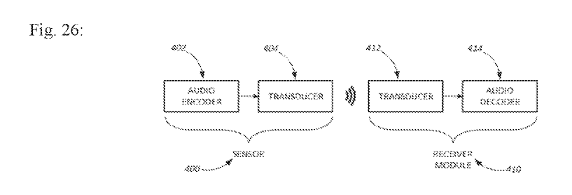

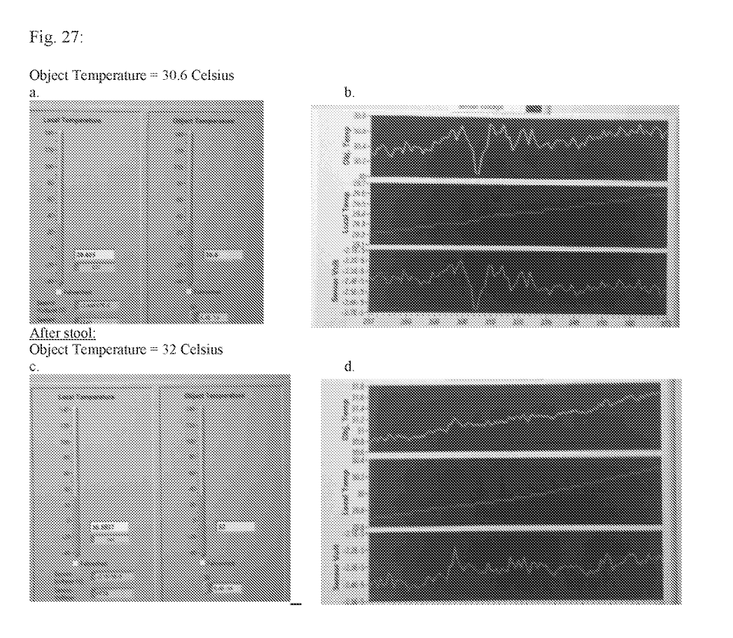

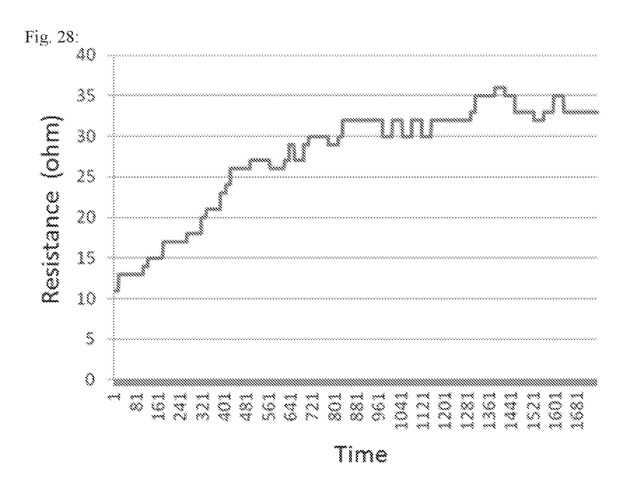

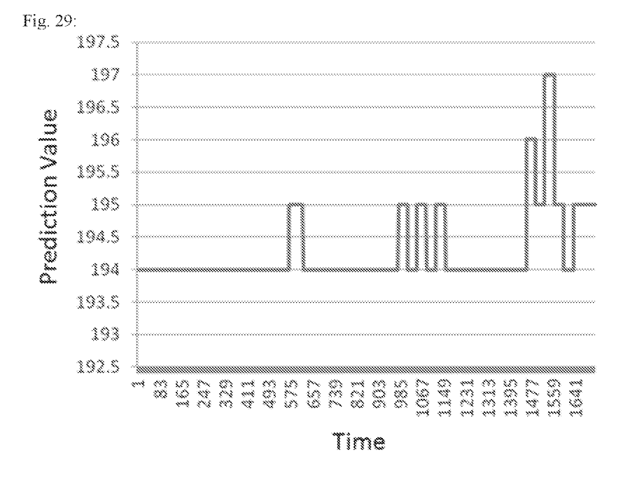

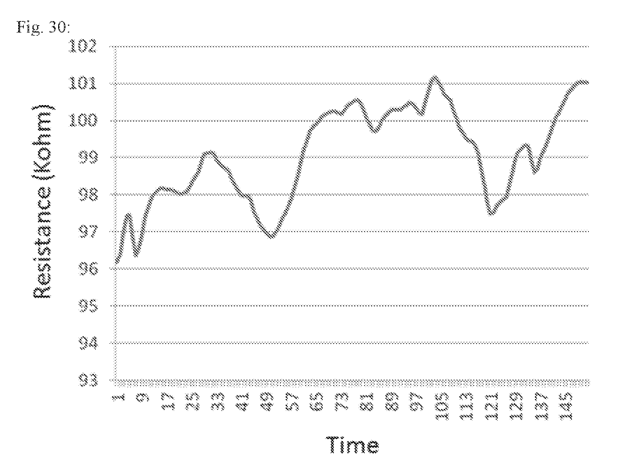

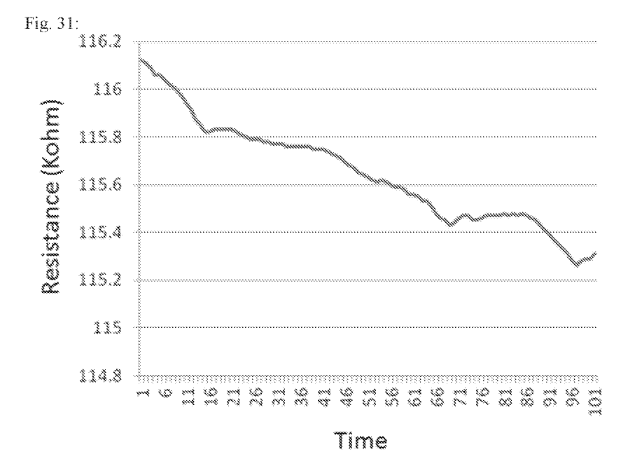

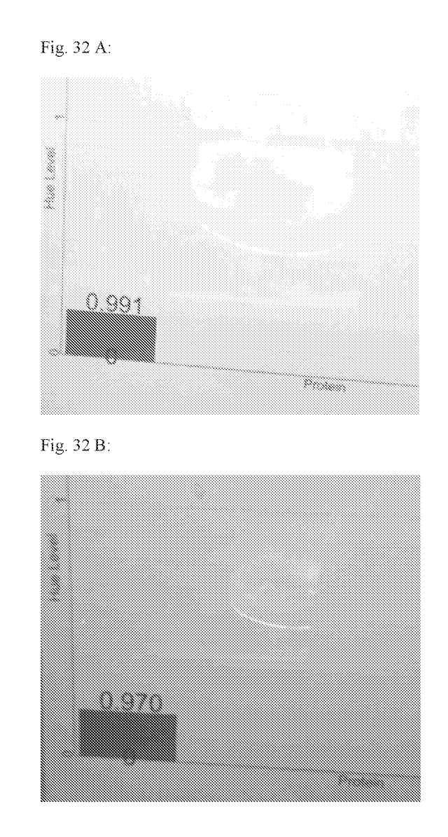

In some embodiments, such as the embodiment shown in FIG. 9, Tube 308 is fluidly connected to sensor 301 and the incontinence device, wherein the tube 308 collects urine from absorbent pad 309, which is placed within the incontinence device 310.

In some embodiments, the housing is attached to an article worn by the subject. The article may be clothing, or, alternatively, the article may be a seat belt, or seatbelt buckle.

Reference is now made to FIG. 10, which shows an embodiment of the chemical test strip 307, comprising discrete test areas for detecting the presence of various analytes in the urine. In the embodiment shown, there is a discrete test area with a reagent that changes color if leukocytes are present in the urine; a discrete test area with a reagent that changes color if nitrite is present in the urine; a discrete test area with a reagent that changes color if urobilinogen is present in the urine; a discrete test area with a reagent that changes color if protein is present in the urine; a discrete test area with a reagent that changes color according to the pH of the urine; a discrete test area with a reagent that changes color according to the specific gravity of the urine; a discrete test area with a reagent that changes color if ketones are present in the urine; a discrete test area with a reagent that changes color if bilirubin is present in the urine; a discrete test area with a reagent that changes color if glucose is present in the urine. In some embodiments, the color change on the discrete test area is proportional to the concentration of the analyte. The choice of the reagent is readily determined by one of ordinary skill in the art.

In some embodiments, the urine analyzer is further configured to detect flatus and/or fecal matter.

In some embodiments, the present invention includes devices and methods useful for detection of flatus, fecal matter, or both flatus and fecal matter. According to certain embodiments, the frequency of flatulence, or the frequency of defecation, or both is an indication of the GI tract health of an individual.

In some embodiments, the detection of flatus, fecal matter, or both flatus and fecal matter is performed using a device which is externally mounted on the incontinence product, and therefore does not come in direct contact with the flatus or fecal matter. In some embodiments, the sensor allows for convenient, non-intrusive detection of flatus, fecal matter, or both flatus and fecal matter. In alternative embodiments, the device is embedded within the diaper itself.

According to some embodiments, the same sensor detects the presence of flatus and fecal matter in the incontinence product.

The terms “diaper”, “incontinence product” and “absorbent incontinence product” may be used here interchangeably, and relate to a wearable product having a layered construction, which allows the transfer and distribution of urine to an absorbent core structure where it is locked in. Similarly, the term “subject” is used here for convenience only, and may relate to any subject, such as an infant, a child, or an adult.

In certain embodiments, the sensor measures changes in the humidity of the air within the incontinence product, which is indicative of the presence of flatus or fecal matter, or both in the incontinence product.

In certain embodiments, the sensor is a humidity sensor. In certain embodiments, the humidity sensor is a capacitive sensor that consists of two electrodes, separated by a dielectric. In the embodiments, changes in humidity are detected by either a change in the capacitance of the dielectric. Non-limiting examples of humidity sensors suitable for use in the present invention include the P/N:HIH6030 sensor sold by Honeywell.

In certain embodiments, the humidity sensor is a resistive sensor that consists of two electrodes, separated by a conductive layer. In the embodiments, changes in humidity are detected by either a change in the conductance of the conductive layer.

In certain embodiments, the sensor is a volatile organic compound sensor. In certain embodiments, the volatile organic compound sensor is a MOS sense element, capable of detecting more than one gaseous compound, including, for example, CO and volatile organic compounds. The presence of a volatile organic compound alters the resistance of the sensor, that generates a signal that can be decoded, using an integrated circuit into a readout of parts per million of a specific volatile organic compound. Non-limiting examples of volatile organic compound sensors suitable for use in the present invention include the AMS P/N: iAQ-core sensor, and the AMS P/N: AS-MLV-P2 sensor.

According to some embodiments, a first sensor detects the presence of flatus, and a second sensor detects the presence of fecal matter in the incontinence product. A schematic of an embodiment of a device utilizing two sensors is shown in FIG. 11.

In certain embodiments, the first sensor is a humidity sensor. In certain embodiments, the first sensor is a volatile organic compound sensor. In certain embodiments, the second sensor is a humidity sensor. In certain embodiments, the second sensor is a volatile organic compound sensor. In certain embodiments, the first sensor detects flatus. In certain embodiments, the first sensor detects fecal matter. In certain embodiments, the second sensor detects flatus. In certain embodiments, the second sensor detects fecal matter.

Reference is now made to FIG. 11, which shows a schematic of device according to certain embodiments. In the embodiments, the first sensor 600 is configured to detect volatile organic compounds. In the embodiments, the second sensor 601 is configured to detect changes in humidity. In the embodiments, both the first and second sensor output an electrical signal that is processed by the operational amplifier 602 through an A/D 608 and a microcontroller 610, which processes the electrical signal from the first and second sensor. In some embodiments, in place of the microcontroller 610, the urine analyzer comprises either a discrete electronic circuit 604 configured to test logic level of the output signal received from the at least one analyte sensor and determining the signal 611, or a voltage comparator 606 configured to compare the signal received from the at least one analyte sensor and to perform comparing to known reference threshold, determining the signal 611.

In the embodiments, based on the detection, the microprocessor 610, or comparator 606 or transistor 604 will transmit a signal specific for flatus, fecal matter, or both. In the embodiments, the signal can be visual, audible, or tactile. In the embodiments, the signal can be displayed on the device, or, alternatively, transmitted to a remote receiver. Reference is now made to FIG. 12, in certain embodiments, the presence of flatus in the incontinence product elicits a decrease in the voltage output of a humidity sensor. In certain embodiments, the decrease is a 350 mv decrease. In certain embodiments, the decrease is a 300 mv decrease. In certain embodiments, the decrease is a 250 mv decrease. In certain embodiments, the decrease is a 200 my decrease. In certain embodiments, the decrease is a 150 mv decrease. In certain embodiments, the decrease is a 100 mv decrease. In certain embodiments, the decrease is a 50 mv decrease. In certain embodiments, the decrease is a 40 mv decrease. In certain embodiments, the decrease is a 30 mv decrease. In certain embodiments, the decrease is a 20 mv decrease. In certain embodiments, the decrease is a 10 mv decrease.

Reference is now made to FIG. 13, in certain embodiments, the presence of fecal matter in the incontinence product elicits an increase in the voltage output of a humidity sensor. In certain embodiments, the increase is a 10 mv increase. In certain embodiments, the increase is a 20 mv increase. In certain embodiments, the increase is a 30 mv increase. In certain embodiments, the increase is a 40 mv increase. In certain embodiments, the increase is a 50 mv increase. In certain embodiments, the increase is a 60 mv increase. In certain embodiments, the increase is a 70 mv increase. In certain embodiments, the increase is a 80 mv increase. In certain embodiments, the increase is a 90 mv increase. In certain embodiments, the increase is a 100 mv increase.

Reference is now made to FIG. 14, in certain embodiments, the presence of fecal matter in the incontinence product elicits a decrease in resistance of a volatile organic compound sensor. In certain embodiments, the decrease is a 200 ohm decrease. In certain embodiments, the decrease is a 150 ohm decrease. In certain embodiments, the decrease is a 100 ohm decrease. In certain embodiments, the decrease is a 50 ohm decrease. In certain embodiments, the decrease is a 40 ohm decrease. In certain embodiments, the decrease is a 30 ohm decrease. In certain embodiments, the decrease is a 20 ohm decrease. In certain embodiments, the decrease is a 10 ohm decrease.

Reference is now made to FIG. 15, in certain embodiments, the presence of fecal matter in the incontinence product elicits increase in the prediction value of a volatile organic compound sensor. In certain embodiments, the increase is a value of 10. In certain embodiments, the increase is a value of 20. In certain embodiments, the increase is a value of 30. In certain embodiments, the increase is a value of 40. In certain embodiments, the increase is a value of 50. In certain embodiments, the increase is a value of 100. In certain embodiments, the increase is a value of 150. In certain embodiments, the increase is a value of 200. In certain embodiments, the increase is a value of 250. In certain embodiments, the increase is a value of 300.

### An Alternate Sensor According to Some Embodiments of the Present Invention

In some embodiments, the present invention is a urine analyzer comprising:
d. at least one discrete test area configured to perform a colorimetric test specific for an analyte selected from the group consisting of glucose, protein, pH, blood, ketone bodies, bilirubin, and any combination thereof, wherein each discrete test area is configured to perform a different colorimetric test;
e. at least one color sensor, configured to read the at least one discrete test area, and output an indication based on the presence of the at least one analyte; and
f. an integrated circuit, configured to receive an indication from the at least one color sensor, and configured to compute, based on the indication from the at least one color sensor, the amount of at least one analyte selected from the group consisting of glucose, protein, pH, blood, ketone bodies, bilirubin, specific gravity, urobilinogen, nitrite, leuokocytes, blood, and any combination thereof, in the urine.

In some embodiments, the urine analyzer is further configured to perform an optical analysis of the urine, wherein an optical sensor is configured to measure the absorbance of the urine and output an indication, based on the absorbance, and the integrated circuit is further configured to receive the indication, and configured to compute the concentration of the urine.

In some embodiments, the at least one discrete test area is incorporated into a test strip.

In some embodiments, the test strip may be removed from the urine analyzer and disposed of.

In some embodiments, every discrete test area has a single color sensor positioned over it, and each color sensor is configured to output an indication based on the presence of the at least one analyte.

In some embodiments, the integrated circuit is further configured to compute, based on the indication from the color sensors positioned over every discrete test area, the amount of at least one analyte selected from the group consisting of glucose, protein, pH, blood, ketone bodies, bilirubin, specific gravity, urobilinogen, nitrite, leuokocytes, blood, and any combination thereof, in the urine.

In some embodiments, the urine analyzer is configured to be integrated into an incontinence device.

In some embodiments, the diaper is configured to allow urine to exit the diaper and enter the urine analyzer.

In some embodiments, the diaper comprises a connector that attaches the urine analyzer to the diaper, and the connector has an orifice, forming a channel through the diaper, that allows urine to exit the diaper and enter the urine analyzer, without being absorbed by the diaper.

In some embodiments, the connector is integrated into the diaper during the manufacturing process of the diaper.

In some embodiments, the connector us added to the diaper after the diaper has been manufactured. In some embodiments, the connector is disposable.

In some embodiments, the diaper is worn by a subject.

In some embodiments, the urine analyzer is configured to be attached to an article used by a subject.

In some embodiments, the article configured to be used by the subject is selected from the group consisting of bedding, and a seat belt latch.

In some embodiments, the subject is an infant.

In some embodiments, the subject is an adult.

In some embodiments, the integrated circuit is configured to transmit the computation to a remote device.

In some embodiments, the integrated circuit is configured to transmit the computation to a remote device via a radio transmission.

In some embodiments, the integrated circuit is configured to transmit the computation to a remote device via an audible signal.

In some embodiments, the integrated circuit is configured to transmit the computation to a remote device via a tactile signal.

In some embodiments, the integrated circuit is configured to transmit the computation to a remote device via a visual signal.

In some embodiments, the urine analyzer further comprises a screen operatively coupled to the integrated circuit, wherein the screen is configured to display the computation.

In some embodiments, urine analyzer further comprises at least one light-emitting diode (LED) configured to illuminate the at least one discrete area of the test strip.

In some embodiments, the at least one LED is a white-light LED.

In some embodiments, the at least one LED is an infrared LED.

In some embodiments, the present invention is a urine analyzer comprising:
d. at least one discrete test area configured to perform a colorimetric test specific for an analyte selected from the group consisting of glucose, protein, pH, blood, ketone bodies, bilirubin, and any combination thereof, wherein each discrete test area is configured to perform a different colorimetric test;
e. at least one color sensor, configured to read the at least one discrete test area, and output an indication based on the presence of the at least one analyte; and
f. an integrated circuit, configured to receive an indication from the at least one color sensor, and compute, based on the indication from the at least one color sensor, the amount of at least one analyte selected from the group consisting of glucose, protein, pH, blood, ketone bodies, bilirubin, specific gravity, urobilinogen, nitrite, leuokocytes, blood, and any combination thereof, in the urine.

In some embodiments, the urine analyzer is further configured to perform an optical analysis of the urine, wherein an optical sensor is configured to measure the absorbance of the urine and output an indication, based on the absorbance, and the integrated circuit is further configured to receive the indication, and compute the concentration of the urine.

In some embodiments, the at least one discrete test area is incorporated into a test strip.

In some embodiments, the test strip may be removed from the urine analyzer and disposed of.

In some embodiments, every discrete test area has a single color sensor positioned over it, and each color sensor is configured to output an indication based on the presence of the at least one analyte.

In some embodiments, the integrated circuit is further configured to compute, based on the indication from the color sensors positioned over every discrete test area, the amount of at least one analyte selected from the group consisting of glucose, protein, pH, blood, ketone bodies, bilirubin, specific gravity, urobilinogen, nitrite, leuokocytes, blood, and any combination thereof, in the urine.

In some embodiments, the urine analyzer is further configured to locate the integrated circuit remotely from the at least one discrete test area, and the at least one color sensor.

In some embodiments, the urine analyzer is further configured to locate the integrated circuit and the at least one color sensor remotely from the at least one discrete test area.

In some embodiments, the at least one discrete test area, and the at least one color sensor are attached to, or integrated into the inside surface of a diaper, and the integrated circuit is attached to, or integrated into the outside surface of the diaper.

In some embodiments, the integrated circuit is connected to the components of the urine analyzer that are located remotely by a connecting cable.

In some embodiments, the urine analyzer is configured to be integrated into an incontinence device.

In some embodiments, the urine analyzer is configured to be attached to an incontinence device.

In some embodiments, the urine analyzer is configured to be attached to an article worn by the subject.

In some embodiments, the incontinence device is an absorbent pad.

In some embodiments, the absorbent pad is placed under the subject.

In some embodiments, the absorbent pad is placed on the subject's bedding.

In some embodiments, the subject is an infant.

In some embodiments, the subject is an adult.

In some embodiments, the integrated circuit is configured to transmit the computation to a remote device.

In some embodiments, the integrated circuit is configured to transmit the computation to a remote device via a radio transmission.

In some embodiments, the integrated circuit is configured to transmit the computation to a remote device via an audible signal.

In some embodiments, the integrated circuit is configured to transmit the computation to a remote device via a tactile signal.

In some embodiments, the integrated circuit is configured to transmit the computation to a remote device via a visual signal.

In some embodiments, the urine analyzer further comprises a screen operatively coupled to the integrated circuit, wherein the screen is configured to display the computation.

In some embodiments, urine analyzer further comprises a light-emitting diode (LED) configured to illuminate the at least one discrete area of the test strip.

In some embodiments, the at least one LED is a white-light LED.

In some embodiments, the at least one LED is an infrared LED.

In some embodiments, the present invention includes devices and methods useful for collecting and analyzing urine from a subject. According to certain embodiments, the frequency of urination, the volume of urine, the hydration level, the levels of at least one analyte selected from the group consisting of glucose, protein, pH, blood, ketone bodies, bilirubin, specific gravity, urobilinogen, nitrite, leuokocytes, blood, and any combination thereof, is an indication of the health of the subject.

In some embodiments, the present invention is a urine analyzer comprising: a sensor comprising: (i) at least one discrete test area configured to perform a colorimetric test specific for an analyte selected from the group consisting of glucose, protein, pH, blood, ketone bodies, bilirubin, and any combination thereof, wherein each discrete test area is configured to perform a different colorimetric test; (ii) at least one color sensor, configured to read the at least one discrete test area, and output an indication based on the presence of the at least one analyte; and (iii) an integrated circuit, configured to receive an indication from the at least one color sensor, and compute, based on the indication from the at least one color sensor, the amount of at least one analyte selected from the group consisting of glucose, protein, pH, blood, ketone bodies, bilirubin, specific gravity, urobilinogen, nitrite, leuokocytes, blood, and any combination thereof, in the urine.

In some embodiments, the urine analyzer is further configured to perform an optical analysis of the urine, wherein an optical sensor is configured to measure the absorbance of the urine and output an indication, based on the absorbance, and the integrated circuit is further configured to receive the indication, and compute the concentration of the urine.

In some embodiments, the at least one discrete test area is incorporated into a test strip.

In some embodiments, the test strip may be removed from the urine analyzer and disposed of.

In some embodiments, every discrete test area has a single color sensor positioned over it, and each color sensor is configured to output an indication based on the presence of the at least one analyte.

In some embodiments, the integrated circuit is further configured to compute, based on the indication from the color sensors positioned over every discrete test area, the amount of at least one analyte selected from the group consisting of glucose, protein, pH, blood, ketone bodies, bilirubin, specific gravity, urobilinogen, nitrite, leuokocytes, blood, and any combination thereof, in the urine.

A urine analyzer according to some embodiments is shown in FIGS. 16 and 17. In the embodiment shown in FIG. 16, urine analyzer 3 is configured to be attached to a diaper.

Referring to FIGS. 17 A and B, in some embodiments, urine analyzer 3 comprises a bottom adhesive layer 4, onto which is attached a printed circuit board 1, containing a plurality of color sensors 2, white light LED's 5, which illuminate the portion of a support 8, containing the plurality of discrete chemical test areas 11. The printed circuit board 1 contains a power source 6, and microcontroller 7. A transparent impermeable layer 9 is located on top of the printed circuit board 1, onto which is located support 8. A top layer 10 is located on top of the support 8. In some embodiments, the top layer 10 is absorbent.

In some embodiments, the electronic components of the urine analyzer are waterproof.

FIGS. 16-18 shows a urine analyzer according to some embodiments of the present invention. FIG. 18 shows the urine analyzer attached to the inside of an incontinence device, and configured to absorb and analyze a portion of any urine that may be deposited within or on to the incontinence device.

In some embodiments, urine analyzer 3 comprises a bottom adhesive layer 4, onto which is attached a printed circuit board 1, containing a plurality of color sensors 2, white light LED's 5, which illuminate the portion of a support 8, containing the plurality of discrete chemical test areas 11. Bottom adhesive layer 4, and printed circuit board 1 are attached to, or integrated into the inside surface of a diaper. Power source 6, and microcontroller 7 are attached to, or integrated into the outside surface of the diaper.

In some embodiments, the power source 6, and microcontroller 7 are connected to the components of the urine analyzer that are located remotely by a connecting cable. Referring to FIGS. 19 to 22, in some embodiments, urine analyzer 3 is further configured to comprise a cable 11, connector 12, which connects urine analyzer 3 to an external device 13. In some embodiments, external device 13 contains power source 6, and microcontroller 7. In some embodiments, external device 13 is further configured to comprise a screen.

In some embodiments, urine analyzer 3 is further configured to comprise a sensors extension 14, with plurality of optical, volatile organic compounds and humidity sensors.

In some embodiments, the optical sensor is a photodetector selected from the group consisting of: photoresistors, photovoltaic cells, photodiodes, infrared sensors, phototransistors, CCDs (charge coupled devices), and CMOS (complementary metal oxide semiconductors). The photodetector detects the shade of the urine, which is known to be indicative of the level of hydration. Generally, the darker the urine, the more dehydrated the subject is, and vice versa. Transparent urine usually means a good hydration level. Optionally, the sensor also measures the amount of urine secreted, so that it estimates the level of hydration using a combination of two factors: amount and shade.

In some embodiments, the photodetector is the photodetector disclosed in PCT Publication No. WO 2014/064680, the contents of which are incorporated by reference in its entirety.

In some embodiments, some, or all of the components of the urine analyzer are reusable.

A urine analyzer according to some embodiments is shown in FIG. 23. In the embodiment shown in FIG. 23, a light source 108 illuminates a plurality of discrete chemical test areas, over which a plurality of color sensors 106, wherein a single color sensor is positioned over a discrete test area in the plurality of discrete test areas, and each color sensor is configured to output an indication based on the presence of the at least one analyte. The indication is received by a microcontroller 208. The microcontroller 208 is configured to process the output signal from the plurality of color sensors 106, and transmit a signal 209 specific for a specific analyte.

In some embodiments, the signal can be visual, audible, or tactile. In the embodiments, the signal can be displayed on the sensor, or, alternatively, transmitted to a remote receiver or device.

The light source 108 can emit light of any wavelength and intensity. In some embodiments, the light source 108 is a white-light LED. In some embodiments, the light source 108 is an infrared LED.

Non-limiting examples of color sensors include the RGB sensor manufactured by AMS, and has the part number TCS3471, and the RGB sensor manufactured by Intersil, and has the part number ISL29125.

In certain embodiments, the urine analyzer is further configured to include an additional sensor, selected from the group consisting of: a Hall Effect detector, an acoustic detector, a magnetic detector, a color detector, a gyro, a tilt sensor, an accelerometer, and a pressure sensor.

In some embodiments, in place of the microcontroller 208, the urine analyzer comprises either a discrete electronic circuit 204 configured to test logic level of the output signal received from the at least one analyte sensor and determining the signal 209, or a voltage comparator 206 configured to compare the signal received from the at least one analyte sensor and to perform comparing to known reference threshold, determining the signal 209.

Referring to FIG. 24, in some embodiments, the urine analyzer 3 comprises two rows of three discrete chemical test areas 8, each chemical test area specific for a particular analyte. One of ordinary skill in the art an readily appreciate, however, that the urine analyzer can comprise any number of rows of discrete chemical test areas, and any number of discrete chemical test areas.

In the embodiment shown in FIG. 24, the discrete chemical test areas 8 are attached to a strip, the strip having a hole 14 underneath each chemical test area 8, which facilitates absorption of urine.

In some embodiments, the urine analyzer comprises a discrete test area with at least one reagent that changes color when a specific analyte is present. The at least one reagent can perform a specific test, such as an ELISA for an analyte.

In some embodiments, the urine analyzer comprises a discrete test area with at least one reagent that changes color if leukocytes are present in the urine; a discrete test area with at least one reagent that changes color if nitrite is present in the urine; a discrete test area with at least one reagent that changes color if urobilinogen is present in the urine; a discrete test area with at least one reagent that changes color if protein is present in the urine; a discrete test area with at least one reagent that changes color according to the pH of the urine; a discrete test area with at least one reagent that changes color according to the specific gravity of the urine; a discrete test area with at least one reagent that changes color if ketones are present in the urine; a discrete test area with at least one reagent that changes color if bilirubin is present in the urine; a discrete test area with at least one reagent that changes color if glucose is present in the urine; a discrete test area with at least one reagent that changes color if a pathogen is present in the urine. In some embodiments, the color change on the discrete test area is proportional to the concentration of the analyte. The choice of the reagent is readily determined by one of ordinary skill in the art.

In some embodiments, the diaper is configured to allow urine to exit the diaper and enter the urine analyzer.

Referring to FIG. 25 A, in some embodiments, the sensor comprises an enclosure 23, containing a printed circuit board 20, comprising at least one color sensor 21, wherein a light guide 22 is positioned over the at least one color sensor 21. The enclosure 23 also contains at least one LED light 24, configured to illuminate the diaper. The diaper comprises a reagent cartridge 26, which is attached to the diaper via a connector 29. The reagent cartridge 26 has at least one reagent pad 25 positioned under the at least one color sensor 21 and light guide 22.

In some embodiments, the reagent cartridge 26 is disposable. In some embodiments, the reagent cartridge 26 is attached to the surface 27 of the diaper. In some embodiments, the reagent cartridge 26 is incorporated into the absorbent material 28 of the diaper. In some embodiments, the reagent cartridge 26 is incorporated into the enclosure 23.

In some embodiments, the connector 29 is added to the diaper after the diaper has been manufactured. In some embodiments, the connector 29 is disposable.

Referring to FIG. 2 A, in some embodiments, the reagent cartridge is attached to the external surface of urine collection bag. In some embodiments, the reagent cartridge is incorporated into the enclosure of the urine analyzer (see FIG. 2 B).

Communication with External and Remote Devices According to Some Embodiments of the Present Invention

According to some embodiments, the urine analyzer disclosed herein may further interface and/or communicate with an external and/or remote device to convey a signal generated by the urine analyzer disclosed herein to the external and/or remote device (herein, a “receiver” or a “receiving device”). Conveying the signal from the urine analyzer to the receiving device may be performed by various communication routes, such as radio frequency or acoustic communication.

The communication between the urine analyzer and the external and/or remote device can be bi-directional.

Acoustic communication makes use of sound and/or ultrasound, whereby a “transmitter” produces a sound that is detected by a “receiver”. Sound is produced by the transmitter when a physical object vibrates rapidly, disturbs nearby air molecules (or other surrounding medium) and generates compression waves that travel in all directions away from the source. Sound can be made to vary in frequency (high pitch vs. low pitch), amplitude (loudness), and periodicity (the temporal pattern of frequency and amplitude). Since acoustic waves move rapidly through the medium, acoustic signals can be quickly started, stopped, or modified to send a time-sensitive message.

According to some embodiments, for each of the various physiological conditions detected by the sensing devices and systems as disclosed herein, a different acoustical signal may be generated by one or more transducers connected to the microcontroller. The various acoustical signals may differ by various parameters, such as, but not limited to: frequency, periodicity, amplitude, duration, series of signals and the intervals therebetween (duty cycle) and/or the like. The frequency of the acoustic alert may be in any range. In an embodiment, the acoustic alert is in the range of 1 Hz to 10 KHz. In another embodiment, the acoustic alert is in the range of 10 Khz to 18 Khz. In another embodiment, the acoustic alert is in the range of 18 KHz to 20 Khz. In another embodiment, the acoustic alert is in the range of 18 KHz to 22 Khz. In another embodiment, the acoustic alert is in the range of 20 KHz to 22 Khz. In another embodiment, the acoustic alert is higher in the ultrasonic range, such as above 22 KHz.

For example, if the urine analyzer detects urine, it may produce an 8 KHz tone, optionally in conjunction with other series of tones. For example, if the urine analyzer detects feces, it may produce an 8 KHz tone, optionally in conjunction with other series of tones. As another example, if the urine analyzer detects high temperature it may produce a 5 Khz tone, optionally in conjunction with other series tones. As yet a further example, if the urine analyzer detects a breathing problem, it may produce a 20 Khz tone, optionally in conjunction with other series of tones. These were simplistic examples, meant merely to demonstrate how acoustic communication may be realized.

According to further embodiments, the acoustical signal produced by the urine analyzer may be received by a receiving device, which is equipped with a microphone. Various acoustic communication protocols may be used for establishing an acoustic communication between the transmitter (the urine analyzer) and the receiving device.

Non-limiting examples of communication protocols are disclosed in WO 2014/064680, the contents of which are incorporated by reference in its entirety.

According to some embodiments, the receiving device may include any type of device configured to receive an acoustic signal via the appropriate acoustic communication protocol, and may further convey the signal to a user, who may be located in a remote location. An added benefit of such a setting is that acoustic communication, unlike radio frequency communication, does not involve electromagnetic radiation in the subject's area, thereby increasing the safety of use of the devices and systems disclosed herein.

According to some embodiments, the acoustic tone or set of tones which may be generated by the urine analyzer define an acoustic protocol in the time domain. In some embodiments, the protocol may be programmed in the urine analyzer's microcontroller and in the receiving device.

According to some embodiments, an exemplary acoustic protocol may include the following “packets”: (1) start bit, get ready for tone sequence; (2) first tone; (3) second tone; (4) Nth tone; (5) stop bit, tone sequence stopped. Any of the steps and the time length, number of bits and frequency of the bit tone, loops, and the like, may be changed to define an appropriate protocol.

Reference is now made to FIG. 26, which schematically illustrates an exemplary acoustic communication interface between urine analyzer 400 and a receiver module 410. Urine analyzer 400 includes an audio encoder 402, adapted to produce an acoustic signal based on the signal produced by the urine analyzer. Audio encoder 402 may be incorporated in the microcontroller discussed earlier, or be connected to it. The urine analyzer further includes a transducing element 404, adapted to convert an electrical signal from audio encoder 402 into an acoustic signal transmitted towards the remote receiver. In some embodiments, the transducing element 404 is a speaker. The acoustic signal produced by the urine analyzer may then be detected by transducer unit 412 of receiving module 410. In some exemplary embodiments, transducer 412 is a microphone. The acoustic signal may then be decoded by audio decoder 414 of the receiving device. Decoding the acoustic signal may be used to convert the acoustic signal to an electrical signal. The decoded signal may be processed and conveyed to a user. In some embodiments, the decoded signal may be converted to an alarm signal that may a visual signal, a tactile signal, an audible signal, and the like, or any combination thereof.

In some embodiments, the urine analyzer of the present invention plays music, which also serves as a medium for transmitting the acoustic signal. Namely, audio decoder 414 at receiving module 410 may be configured to decode certain music or tones played by the bag and attribute it to a displacement event. In some other embodiments, the acoustic signal may be separate from the music or the sound played, whether by superimposing the acoustic signal on the music, or by transmitting the acoustic signal at a different time than the music.

Non-limiting examples of devices capable of decoding an acoustic signal transmitted by the sensor are disclosed in U.S. patent application Ser. No. 14/704,443, the contents of which are incorporated by reference in its entirety.

According to some embodiments, the receiving device may be portable. In some embodiments, the receiving device may be placed in the vicinity of the sensing device. In some embodiments, the receiving device may be place at a remote location, but still in acoustic communication range from the transmitting device. In some exemplary embodiments, the receiving device is a smart phone. In some exemplary embodiments, the receiving device is configured to communicate with a smart phone.

In some embodiments, the term “smart phone” may refer to any portable electronic device. For example, a smart phone can include, but is not limited to, a mobile phone, Personal Digital Assistant (PDA), Blackberry™, Pager, Smartphone, or any other reasonable mobile electronic device. For ease, at times the above variations are not listed or are only partially listed, this is in no way meant to be a limitation.

In some embodiments, when an event is detected by the urine analyzer, an acoustic alert is produced by the urine analyzer. The acoustic alert is detected by a receiving device such as receiver module 410, which is located in the proximity of the subject. The receiving device may then issue an alert (such as audible, tactile and/or visual alert) to a supervisor. Additionally or alternatively, the receiving device may serve as a relay station configured to communicate with a remote device (such as smart phone 302), which is, in turn, configured to generate an appropriate alarm to the supervisor.

In some embodiments, the receiving device is configured to communicate with the remote device via the Internet and/or via short-range radio, utilizing technologies such as WiFi, Bluetooth, SMS, cellular data communication, push notification protocol, and activate the alarm therein, in order to notify a supervisor which may be located in a remote location. The remote device may execute an application for communicating with the receiving device and to produce audible and/or visual alarm and/or tactile alarms.

In some embodiments, the receiving device is an Apple iPhone 4 smart phone (hereinafter “iPhone”). The iPhone's microphone can pick up the acoustic signals emitted by the sensing device, and then transmit a signal via Apple's push notification service (APN). A non-limiting example is shown in FIG. 1.

In some embodiments, Apple Push Notification service is intended to relay messages to iDevices even when a target application on the receiving device is not running. The APN transports and routes a notification from a given provider to a given device. A notification is a short message consisting of two major pieces of data: the device token and the payload. The device token contains information that enables the APN to locate the device on which the client application is installed. The APN also uses it to authenticate the routing of a notification. The payload is a JSON-defined property list that specifies how the user of an application on a device is to be alerted. The flow of remote-notification data is one-way. The provider composes a notification package that includes the device token for a client application and the payload. The provider sends the notification to the APN which, in turn, pushes the notification to the device.

More details on Apple's Push Notification service and related issues is available online at Apple's iOS Developer Library, http://developer.apple.com/library/ios/navigation/, which is incorporated herein by reference in its entirety.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

Reference is now made to the following examples, which together with the above descriptions illustrate some embodiments of the invention in a non-limiting fashion.

### EXAMPLES

### Example 1: Detection of Feces Using a Device According to Some Embodiments of the Present Invention

A sensor containing a TMP006 thermopile was attached to the external surface of a diaper worn by an adult subject. A baseline recording was obtained for 2 minutes. After this time, the subject defecated, and the recording was continued for an additional 4 minutes. The stool was small and located approximately 1 cm away from the sensor. Nevertheless, the thermopile detected the thermal radiation emitted by the stool. See FIG. 27, panel c (showing an increase in temperature).

### Example 2: Detection of Flatus and Fecal Matter in an Incontinence Product Worn by an Adult Subject, Using a Device Containing a Humidity Sensor

A 37 year old adult human subject wore an incontinence product, with a device containing a humidity sensor (HIH6030, Honeywell) attached. The sensor recorded humidity levels in the diaper over time, and reported the changes in humidity levels observed when the human subject defecated and passed flatus. The results are shown in FIGS. 12 and 13.

Referring to FIG. 12, a decrease in voltage from approximately 250 mv to approximately 0 mv was observed at time 10, when the adult subject passed flatus.

Referring to FIG. 13, an increase in voltage from approximately 70 mv to approximately 87 mv was observed at time 10, when the adult subject defecated. Taken together, these data demonstrate that a humidity sensor is capable of reporting an unsoiled incontinence product (e.g., the signal recorded before time 10), and the presence of flatus and/or fecal matter in the incontinence product (e.g., a decrease in voltage, or an increase in voltage, respectively).

### Example 3: Detection of Flatus and Fecal Matter in an Incontinence Product Worn by an Adult Subject, Using a Device Containing a Volatile Organic Compound Sensor

A 3 year old human subject wore an incontinence product, with a device containing a volatile organic compound sensor (AMS P/N: iAQ-core) attached. The sensor recorded volatile organic compound levels in the diaper, and reported the changes in (i) resistance (FIGS. 14 and 28); (ii) prediction (FIGS. 15 and 29) over time, when the diaper was clean (FIGS. 28 and 29), and when the infant defecated (FIGS. 14 and 15).

Referring to FIG. 14, a decrease in resistance from 117 ohm to 50 ohm was observed when the subject defecated.

Referring to FIG. 15, an increase in the prediction value of 140 to 260 was observed when the subject defecated. The 260 prediction value was calculated to be the presence of volatile organic compounds at a concentration of 988 ppm, using the data sheet provided by the sensor manufacturer.

Referring to FIG. 28, an increase in resistance from 12 ohm to 33 ohm was observed.

Referring to FIG. 29, no change in the prediction value was observed. The prediction value remained at approximately 195, which was calculated to be the presence of volatile organic compounds at a concentration of 450 ppm.

### Example 4: Detection of Flatus and Fecal Matter in an Incontinence Product Worn by an Adult Subject, Using a Device Containing an Alternative Volatile Organic Compound Sensor

A 3 year old human subject wore an incontinence product, with a device containing a volatile organic compound sensor (AMS P/N: AS-MLV-P2) attached. The sensor recorded volatile organic compound levels in the diaper, and reported the changes in (i) resistance when the infant defecated (FIG. 30); and resistance over time in a clean diaper (FIG. 31).

Referring to FIG. 30, an average resistance value of 98 Kohm was reported in a diaper containing fecal matter, which was calculated to be a volatile organic compound concentration of 50 ppm. In contrast, in a clean diaper, resistance levels in the range of 116 Kohm to 115 kOhm were observed, which was calculated to be a volatile organic compound concentration of 25 ppm.

### Example 5: Detection of Protein in an Incontinence Product Worn by an Adult Subject, Using a Device Containing a Reagent Sensor

A 38 year old human subject wore an incontinence product, with a device containing a urine color reagent sensor. The sensor recorded protein reagent color change in the diaper, and reported the changes in decimal value when the adult urinated (FIG. 32 A); and decimal value over time in a clean diaper (FIG. 32 B).

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

What is claimed is:

1. A urine analyzer, comprising:
a urine collection bag; and
a sensor, comprising:
a. at least one light-emitting diode (LED) configured to illuminate a portion of the urine collection bag;
b. at least one photodetector configured to output an indication of an amount of light reflected from the portion of the urine collection bag illuminated by the at least one LED; and
c. an integrated circuit, configured to receive an indication from the at least one photodetector, and configured to compute, based on the indication from the at least one photodetector, a volume of urine in the urine collection bag.

2. The urine analyzer of claim 1, configured to be attached to an article configured to be worn by a subject.

3. The urine analyzer of claim 2, wherein the article configured to be worn by the subject is a diaper.

4. The urine analyzer of claim 1, configured to be attached to a subject.

5. A urine analyzer, comprising:
a urine collection bag; and
a sensor, comprising:
i. at least one light-emitting diode (LED) configured to illuminate a portion of the urine collection bag;
j. at least one photodetector configured to output an indication of an amount of light reflected from the portion of the urine collection bag illuminated by the at least one LED;
k. a first analyte sensor configured to detect at least one analyte selected from the group consisting of glucose, protein, pH, blood, ketone bodies, bilirubin, and any combination thereof, and output an indication based on the presence of the at least one analyte; and
l. an integrated circuit, configured to receive an indication from the at least one photodetector and the first analyte sensor, and
i. configured to compute, based on the indication from the at least one photodetector, a volume of urine in the urine collection bag; and
ii. configured to compute, based on the indication from the first analyte sensor, the amount of at least one analyte selected from the group consisting of glucose, protein, pH, blood, ketone bodies, bilirubin, and any combination thereof, in the urine.

6. The urine analyzer of claim 5, configured to be attached to an article configured to be worn by a subject.

7. The urine analyzer of claim 6, wherein the article configured to be worn by the subject is a diaper.

8. The urine analyzer of claim 5, configured to be attached to a subject.

9. The urine analyzer of claim 5, wherein, urine analyzer further comprises:
e. at least one chemical test strip configured to detect at least one analyte selected from the group consisting of glucose, protein, pH, blood, ketone bodies, bilirubin, and any combination thereof; and
f. a second analyte sensor configured to read the at least one chemical test strip, and output an indication based on the presence of the at least one analyte, and wherein the integrated circuit is configured to receive an indication from the at least one photodetector, the first analyte sensor, and the second analyte sensor, and
i. configured to compute, based on the indication from the at least one photodetector, the hydration level of the subject; and
ii. configured to compute, based on the indication from the first analyte sensor and/or the second analyte sensor, the amount of at least one analyte selected from the group consisting of glucose, protein, pH, blood, ketone bodies, bilirubin, and any combination thereof, in the urine.

10. A urine analyzer, comprising:
a urine collection bag; and
a sensor comprising:
g. a fluid reservoir;
h. at least one light-emitting diode (LED) configured to illuminate a portion of the fluid reservoir;
i. at least one photodetector configured to output an indication of an amount of light reflected from the portion of the fluid reservoir illuminated by the at least one LED;
j. at least one chemical test strip configured to detect at least one analyte selected from the group consisting of glucose, protein, pH, blood, ketone bodies, bilirubin, and any combination thereof;
k. a second analyte sensor configured to read the at least one test strip, and output an indication based on the presence of the at least one analyte; and
l. an integrated circuit, configured to receive an indication from the at least one photodetector and the second analyte sensor, and
i. configured to compute, based on the indication from the photodetector, the hydration level of the subject, and
ii. configured to compute, based on the indication from the second analyte sensor, the amount of at least one analyte selected from the group consisting of glucose, protein, pH, blood, ketone bodies, bilirubin, and any combination thereof, in the urine.

11. The urine analyzer of claim 10, configured to be attached to an article configured to be worn by a subject.

12. The urine analyzer of claim 11, wherein the article configured to be worn by the subject is a diaper.

13. The urine analyzer of claim 10, configured to be attached to a subject.

14. A urine analyzer, comprising:
k. a fluid reservoir;
l. at least one light-emitting diode (LED) configured to illuminate a portion of the fluid reservoir;
m. at least one photodetector configured to output an indication of an amount of light reflected from the portion of the fluid reservoir illuminated by the at least one LED;
n. a first analyte sensor configured to detect at least one analyte selected from the group consisting of glucose, protein, pH, blood, ketone bodies, bilirubin, and any combination thereof, and output an indication based on the presence of the at least one analyte; and
o. an integrated circuit, configured to receive an indication from the at least one photodetector and the first analyte sensor, and
i. configured to compute, based on the indication from the at least one photodetector, the hydration level of the subject, and
ii. configured to compute, based on the indication from the first analyte sensor, the amount of at least one analyte selected from the group consisting of glucose, protein, pH, blood, ketone bodies, bilirubin, and any combination thereof, in the urine.

15. The urine analyzer of claim 14, configured to be attached to an article configured to be worn by a subject.

16. The urine analyzer of claim 15, wherein the article configured to be worn by the subject is a diaper.

17. The urine analyzer of claim 14, configured to be attached to a subject.

18. The urine analyzer of claim 14, configured to be attached to an article used by a subject.

19. The urine analyzer of claim 19, wherein the article configured to be used by the subject is selected from the group consisting of bedding, and a seat belt latch.

20. The urine analyzer of claim 14, wherein the urine analyzer further comprises:
e. at least one chemical test strip configured to detect at least one analyte selected from the group consisting of glucose, protein, pH, blood, ketone bodies, bilirubin, and any combination thereof; and
f. a second analyte sensor configured to read the at least one chemical test strip, and output an indication based on the presence of the at least one analyte, and wherein the integrated circuit is configured to receive an indication from the photodetector, the first analyte sensor, and the second analyte sensor, and
i. configured to compute, based on the indication from the at least one photodetector, the hydration level of the subject; and
ii. configured to compute, based on the indication from the first analyte sensor and/or the second analyte sensor, the amount of at least one analyte selected from the group consisting of glucose, protein, pH, blood, ketone bodies, bilirubin, and any combination thereof, in the urine.

21. A urine analyzer, comprising:
m. a fluid reservoir;
n. at least one light-emitting diode (LED) configured to illuminate a portion of the fluid reservoir;
o. at least one photodetector configured to output an indication of an amount of light reflected from the portion of the fluid reservoir illuminated by the at least one LED;
p. at least one chemical test strip configured to detect at least one analyte selected from the group consisting of glucose, protein, pH, blood, ketone bodies, bilirubin, and any combination thereof;
q. a second analyte sensor configured to read the at least one test strip, and output an indication based on the presence of the at least one analyte; and
r. an integrated circuit, configured to receive an indication from the at least one photodetector and the second analyte sensor, and
v. configured to compute, based on the indication from the at least one photodetector, the hydration level of the subject, and
vi. configured to compute, based on the indication from the second analyte sensor, the amount of at least one analyte selected from the group consisting of glucose, protein, pH, blood, ketone bodies, bilirubin, and any combination thereof, in the urine.

22. The urine analyzer of claim 21, configured to be attached to an article configured to be worn by a subject.

23. The urine analyzer of claim 22, wherein the article configured to be worn by the subject is a diaper.

24. The urine analyzer of claim 21, configured to be attached to a subject.

25. The urine analyzer of claim 21, configured to be attached to an article used by a subject.

26. The urine analyzer of claim 25, wherein the article configured to be used by the subject is selected from the group consisting of bedding, and a seat belt latch.

27. The urine analyzer of claim 21, wherein the urine analyzer further comprises at least one sensor comprising a thermopile, configured to output an indication of the presence of urine and/or feces by detecting the thermal radiation emitted by the urine and/or feces, and wherein the integrated circuit is configured to receive an indication from the photodetector, the first analyte sensor, the second analyte sensor, and the thermopile and
vii. configured to compute, based on the indication from the at least one photodetector, the hydration level of the subject;
viii. configured to compute, based on the indication from the second analyte sensor, the amount of at least one analyte selected from the group consisting of glucose, protein, pH, blood, ketone bodies, bilirubin, and any combination thereof, in the urine; and
ix. configured to compute, based on the indication from the at least one sensor comprising a thermopile, the presence of urine and/or feces.

28. A device for detecting flatus, comprising:
a sensor configured to detect flatus and output an indication that flatus has been detected; and
an integrated circuit configured to:
d. receive the indication from the sensor;
e. compute, based on the indication, whether flatus is present in the incontinence product, when the flatus was deposited in the incontinence product; and
f. transmit the computation to a data display unit.

29. The urine analyzer of claim 28, configured to be attached to an article configured to be worn by a subject.

30. The urine analyzer of claim 29, wherein the article configured to be worn by the subject is a diaper.

31. The urine analyzer of claim 28, configured to be attached to a subject.

32. The urine analyzer of claim 28, configured to be attached to an article used by a subject.

33. The urine analyzer of claim 32, wherein the article configured to be used by the subject is selected from the group consisting of bedding, and a seat belt latch.

34. A device for detecting fecal matter, comprising:
a sensor configured to detect fecal matter and output an indication that fecal matter has been detected; and
an integrated circuit configured to:
d. receive the indication from the sensor;
e. compute, based on the indication, whether fecal matter is present in the incontinence product, when the fecal matter was deposited in the incontinence product; and
f. transmit the computation to a data display unit.

35. The urine analyzer of claim 34, configured to be attached to an article configured to be worn by a subject.

36. The urine analyzer of claim 35, wherein the article configured to be worn by the subject is a diaper.

37. The urine analyzer of claim 34, configured to be attached to a subject.

38. The urine analyzer of claim 34, configured to be attached to an article used by a subject.

39. The urine analyzer of claim 38, wherein the article configured to be used by the subject is selected from the group consisting of bedding, and a seat belt latch.

40. A device for detecting flatus and fecal matter, comprising:
a sensor configured to detect flatus and fecal matter and output an indication that flatus and fecal matter has been detected; and
an integrated circuit configured to:
d. receive the indication from the sensor;
e. compute, based on the indication, whether flatus, or fecal matter, or both is present in the incontinence product, when the flatus, or fecal matter was deposited in the incontinence product; and
f. transmit the computation to a data display unit.

41. The urine analyzer of claim 40, configured to be attached to an article configured to be worn by a subject.

42. The urine analyzer of claim 41, wherein the article configured to be worn by the subject is a diaper.

43. The urine analyzer of claim 40, configured to be attached to a subject.

44. The urine analyzer of claim 40, configured to be attached to an article used by a subject.

45. The urine analyzer of claim 44, wherein the article configured to be used by the subject is selected from the group consisting of bedding, and a seat belt latch.

46. A device for detecting flatus and fecal matter, comprising:
a first sensor configured to detect flatus and output an indication that flatus has been detected;
a second sensor configured to detect fecal matter and output an indication that fecal matter has been detected, by detecting signals from the first and second sensor; and
an integrated circuit configured to:
d. receive the indications from the first and second sensor;
e. compute, based on the indications, whether flatus, or fecal matter, or both is present in the incontinence product, when the flatus, or fecal matter was deposited in the incontinence product; and
f. transmit the computation to a data display unit.

47. The urine analyzer of claim 46, configured to be attached to an article configured to be worn by a subject.

48. The urine analyzer of claim 47, wherein the article configured to be worn by the subject is a diaper.

49. The urine analyzer of claim 46, configured to be attached to a subject.

50. The urine analyzer of claim 46, configured to be attached to an article used by a subject.

51. The urine analyzer of claim 50, wherein the article configured to be used by the subject is selected from the group consisting of bedding, and a seat belt latch.

52. A urine analyzer, comprising:
g. at least one discrete test area configured to perform a colorimetric test specific for an analyte selected from the group consisting of glucose, protein, pH, blood, ketone bodies, bilirubin, and any combination thereof, wherein each discrete test area is configured to perform a different colorimetric test;
h. at least one color sensor, configured to read the at least one discrete test area, and output an indication based on the presence of the at least one analyte; and
i. an integrated circuit, configured to receive an indication from the at least one color sensor, and configured to compute, based on the indication from the at least one color sensor, the amount of at least one analyte selected from the group consisting of glucose, protein, pH, blood, ketone bodies, bilirubin, specific gravity, urobilinogen, nitrite, leuokocytes, blood, and any combination thereof, in the urine.

53. The urine analyzer of claim 52, wherein the urine analyzer is further configured to perform an optical analysis of the urine, wherein an optical sensor is configured to measure the absorbance of the urine and output an indication, based on the absorbance, and the integrated circuit is further configured to receive the indication, and configured to compute the concentration of the urine.

54. The urine analyzer of claim 52, wherein the at least one discrete test area is incorporated into a test strip.

55. The urine analyzer of claim 54, wherein the test strip may be removed from the urine analyzer and disposed of.

56. The urine analyzer of claim 42, wherein every discrete test area has a single color sensor positioned over it, and each color sensor is configured to output an indication based on the presence of the at least one analyte.

57. The urine analyzer of claim 52, wherein the integrated circuit is further configured to compute, based on the indication from the color sensors positioned over every discrete test area, the amount of at least one analyte selected from the group consisting of glucose, protein, pH, blood, ketone bodies, bilirubin, specific gravity, urobilinogen, nitrite, leuokocytes, blood, and any combination thereof, in the urine.

58. The urine analyzer of claim 52, wherein the urine analyzer is configured to be integrated into a diaper.

59. The urine analyzer of claim 58, wherein the diaper is configured to allow urine to exit the diaper and enter the urine analyzer.

60. The urine analyzer of claim 59, wherein the diaper comprises a connector that attaches the urine analyzer to the diaper, and the connector has an orifice, forming a channel through the diaper, that allows urine to exit the diaper and enter the urine analyzer, without being absorbed by the diaper.

61. The urine analyzer of claim 52, wherein the urine analyzer is further configured to locate the integrated circuit remotely from the at least one discrete test area, and the at least one color sensor.

62. The urine analyzer of claim 52, wherein the urine analyzer is further configured to locate the integrated circuit and the at least one color sensor remotely from the at least one discrete test area.

63. The urine analyzer of claim 52, wherein the at least one discrete test area, and the at least one color sensor are attached to, or integrated into the inside surface of a diaper, and the integrated circuit is attached to, or integrated into the outside surface of the diaper.

64. The urine analyzer of claim 52, wherein the integrated circuit is connected to the components of the urine analyzer that are located remotely by a connecting cable.

65. The urine analyzer of claim 52, configured to be attached to an article configured to be worn by a subject.

66. The urine analyzer of claim 52, configured to be attached to a subject.

67. The urine analyzer of claim 52, configured to be attached to an article used by a subject.

68. The urine analyzer of claim 67, wherein the article configured to be used by the subject is selected from the group consisting of bedding, and a seat belt latch.
